(19) United States
(12) Patent Application Publication
(10) Pub. No.: US 2026/0272463 A1
(43) Pub. Date: Sep. 17, 2026

(54) LIGHTED SURGICAL STAPLER

(71) Applicant: ENDOLUMIK, INC., MORGANTOWN, WV (US)

(72) Inventors: Nova SZOKA, Morgantown, WV (US); Mara MCFADDEN, Pittsburgh, PA (US)

(21) Appl. No.: 19/168,562

(22) PCT Filed: Mar. 22, 2024
(86) PCT No.: PCT/US2024/021102

§ 371 Date: Sep. 24, 2025

Related U.S. Application Data
(60) Provisional application No. 63/491,991, filed on Mar. 24, 2023.

(51) Int. Cl. A61B 17/115; A61B 17/00; A61B 90/30

(52) U.S. Cl. CPC A61B 17/1155; A61B 90/30; A61B 2017/00907; A61B 2090/306

(57) ABSTRACT

A surgical instrument for treating a tubular organ includes a shaft assembly, a stapling assembly, a trocar, and one or more light sources. The stapling assembly is disposed at the distal end portion of the shaft assembly and includes a circumferential side wall having a distal edge to define a staple line. The trocar is operably movable relative to the stapling assembly along an axis of the stapling assembly between an extended position and a retracted position. The one or more light sources are for emitting light capable of transmitting through a wall of the tubular organ. As such, by detecting light transmitted through the wall of the tubular organ, a user of the surgical instrument can locate the trocar, the distal edge of the stapling assembly or both in the tubular organ.

### CROSS-REFERENCE TO RELATED APPLICATION(S)

The present application claims priority to U.S. Provisional Patent Application No. 63/491,991 filed Mar. 24, 2023. The disclosure of the application is incorporated herein for all purposes by reference in its entirety.

### TECHNICAL FIELD

The present disclosure relates to lighted surgical staplers, and in particular, to lighted circular and/or end-to-end anastomosis surgical staplers for treating tubular organs.

### BACKGROUND

Anastomotic leak (AL) is a life-threatening postoperative complication following colorectal surgery, whose incidence has not substantially decreased over the last 50 years despite advances in anastomotic techniques and perioperative care.

Circular staplers available today are not adequately easy to position, as evidenced by the high leak rates for colorectal anastomoses. It has been reported that anastomotic leaks often constitute the most severe complications and occur in 2.5-36% of surgical cases. These complications are associated with increased length of stay (LOS), higher rates of reoperation, as well as increased morbidity and mortality. The economic burden of these complications is substantial, due to increased LOS and readmissions.

Accordingly, there remains a need for improved devices and methods that enable positioning of the devices within the anatomy.

### SUMMARY

The present disclosure addresses these and other needs in the art by providing circular and/or end-to-end anastomosis surgical instruments that use light (e.g., white and/or near infrared light) to better visualize the location of the intended staple line and trocar within tubular organs (e.g., rectum) before the staples are deployed.

In various embodiments, the present disclosure provides surgical instruments for treating tubular organs of patients. A surgical instrument of the present disclosure includes a shaft assembly, a stapling assembly, a trocar and one or more light sources. The shaft assembly has a distal end portion and a proximal end portion. The stapling assembly is disposed at the distal end portion of the shaft assembly and includes a circumferential side wall having a distal edge to define a staple line. The trocar is disposed at the distal end portion of the shaft assembly and operably movable relative to the stapling assembly along an axis of the stapling assembly between an extended position and a retracted position. When the trocar is in the extended position, the distal tip portion of the trocar is exposed distally relative to the distal edge of the stapling assembly. When the trocar in the retracted position, the distal tip portion of the trocar is concealed proximally relative to the distal edge of the stapling assembly. The one or more light sources are disposed at the trocar, the stapling assembly or both for emitting light that is able to transmit through a wall of the tubular organ of the patient, thereby allowing a user of the surgical instrument to locate the trocar, the distal edge of the stapling assembly or both in the tubular organ of the patient by detecting light transmitted through the wall of the tubular organ.

In some embodiments, the tubular organ is a rectum or colon of the patient.

In some embodiments, the one or more light sources include at least one optic fiber, at least one LED, a polymer containing a near infrared fluorescent material, or any combination thereof. In some embodiments, at least one light source in the one or more light sources emits light in a near-infrared light range. In some embodiments, at least one light source in the one or more light sources is operable to flash, change color, change intensity or any combination thereof.

In some embodiments, at least one light source in the one or more light sources is operable to emit light in any one of a plurality of wavelength ranges. In some such embodiments, the plurality of wavelength ranges includes a first wavelength range and a second wavelength range different from the first wavelength range. In some such embodiments, the first wavelength range is in a white light range and the second wavelength range is in a near-infrared light range.

In some embodiments, the light transmitted through the wall of the tubular organ of the patient is detected by a camera positioned outside of the tubular organ of the patient. In some such embodiments, the light detected by the camera is displayed to provide real-time visualization of where the trocar, the distal edge of the stapling assembly or both are located in the tubular organ of the patient.

In some embodiments, the one or more light sources include a plurality of light sources, wherein light sources in the plurality of light sources are spaced apart at known intervals to serve as markers for detection by a marker-based augmented reality system.

In some embodiments, the stapling assembly further includes an interior base located proximally away from the distal edge and a recess collectively formed by the interior base and the circumferential side wall. In some such embodiments, the one or more light sources includes at least one first light source, at least one second light source, at least one third light source, at least one fourth light source, at least one fifth light source, or any combination thereof. The at least one first light source is disposed at the distal tip portion of the trocar. The at least one second light source is disposed at a proximal base portion of the trocar. The at least one third light source is disposed on the interior base of the stapling assembly. The at least one fourth light source is disposed on an inner surface of the circumferential side wall of the stapling assembly. The at least one fifth light source disposed on an outer surface of the circumferential all of the stapling assembly.

In some embodiments, the at least one first light source is different from the at least one second light source, the at least one third light source, the at least one fourth light source, and/or the at least one fifth light source. In some embodiments, the at least one first light source includes a first light source disposed at a very tip of the trocar. In some embodiments, the at least one first light source, the at least one second light source, or both are disposed inside of the trocar.

In some embodiments, the at least one third light source includes a plurality of third light sources disposed circumferentially around the axis of the stapling assembly on the interior base of the stapling assembly. In some such embodiments, each of the plurality of third light sources is elongated radially with respect to the axis of the stapling assembly. Alternatively, in some embodiments, the at least one third light source consists of a single third light source. In some such embodiments, the single third light source is a ring, encircling the axis of the stapling assembly.

In some embodiments, the at least one fourth light source includes a plurality of fourth light sources disposed circumferentially around the axis of the stapling assembly on the inner surface of the circumferential side wall of the stapling assembly. In some such embodiments, each of the plurality of fourth light sources is elongated along the axis of the stapling assembly. Alternatively, in some embodiments, the at least one fourth light source consists of a single fourth light. In some such embodiments, the single fourth light source is a ring, encircling the axis of the stapling assembly.

In some embodiments, the at least one fifth light source includes a plurality of fifth light sources disposed circumferentially around the axis of the stapling assembly on the outer surface of the circumferential side wall of the stapling assembly. In some such embodiments, each of the plurality of fifth light sources is elongated along the axis of the stapling assembly. Alternatively, in some embodiments, the at least one fifth light source consists of a single fifth light. In some such embodiments, the single fifth light source is a ring, encircling the axis of the stapling assembly.

In some embodiments, the trocar is made of a material transparent and/or translucent to the light emitted by the at least one first light source, the at least one second light source, or both. In some such embodiments, the material is a medical grade plastic. In some embodiments, the medical grade plastic includes acrylic (PMMA), transparent sulfone polymers, polymethyl methacrylate, PVC, polycarbonate, polyethylene terephthalate, polypropylene, polyethylene, styrene methyl methacrylate, or ionomer resin.

In some embodiments, the surgical instrument further includes an anvil removably attached to the trocar. In some such embodiments, at least one light source in the one or more light sources changes color, changes intensity, flashes, or any combination thereof when the anvil is properly attached to the trocar. In some embodiments, the one or more light sources include a light source at the distal tip of the trocar. In some such embodiments, the light source at the distal tip of the trocar is fully concealed by a complete attachment of the anvil to the trocar.

In some embodiments, the surgical instrument further includes a sensor and/or a switch configured to detect the attachment of the anvil to the trocar.

In some embodiments, the surgical instrument further includes a handle assembly. The handle assembly is disposed at the proximal end portion of the shaft assembly for operating the stapling assembly, the trocar, the one or more light sources, or any combination thereof. In some embodiments, the handle assembly includes a battery to power the one or more light sources, to aid the operation of the stapling assembly, to aid the operation of the trocar, or any combination thereof. In some embodiments, the handle assembly is electrically connected to a power outlet to power the one or more light sources, to aid the operation of the stapling assembly, to aid the operation of the trocar, or any combination thereof. In some embodiments, the handle assembly includes a rotatable knob connected to the trocar for extending and/or retracting the trocar.

In some embodiments, the operating of the stapling assembly, the trocar, the one or more light sources, or any combination thereof is powered and/or automatic. Alternatively, in some embodiments, the operating of the stapling assembly, the trocar, the one or more light sources, or any combination thereof is manual via the handle assembly.

In some embodiments, the stapling assembly, the trocar, and the one or more light sources collectively form a stapler head. In some such embodiments, the stapler head is removably attached to the distal end portion of the shaft assembly, disposable or both.

In various embodiments, the present disclosure provides methods for treating tubular organs of patients. A method of the present disclosure includes (A) inserting a stapler head of a surgical instrument into a first segment of the tubular organ. The stapler head of the surgical instrument includes a stapling assembly, a trocar and one or more light sources. The stapling assembly has a distal edge to define a staple line. The trocar is operably movable relative to the stapling assembly along an axis of the stapling assembly. The one or more light sources disposed at the trocar and/or the stapling assembly to emit light capable of transmitting through a wall of the tubular organ.

The method also includes: (B) detecting light transmitted through the wall of the tubular organ to locate the trocar, the distal edge of the stapling assembly or both in the first segment of the tubular organ; (C) determining, based on the detecting (B), whether the distal edge of the stapling assembly is positioned at a desired section of the first segment of the tubular organ; and (D) moving, based on the determining (C), the stapler head of the surgical instrument until the distal edge of the stapling assembly is positioned at the desired section of the first segment of the tubular organ. In some embodiments, the detecting (B) includes: (i) visualization by the user of the surgical instrument, (ii) detection by a laparoscopic and/or robotic camera, or both. In some embodiments, the camera is operable in a white light mode, a near-infrared mode, or both. In some embodiments, the light detected in the detecting (B) is displayed to provide real-time visualization of where the trocar, the distal edge of the stapling assembly or both are located in the tubular organ of the patient.

In some embodiments, the method further includes (E) extending the trocar to puncture the desired section of the first segment of the tubular organ. In some such embodiments, the extending (E) is achieved by rotating a knob at a handle assembly of the surgical instrument in a first direction.

In some embodiments, the method further includes (F) inserting an anvil into a second segment of the tubular organ. The anvil includes a connection portion protruding proximally out of a desired section of the second segment of the tubular organ. The inserting (F) may be performed prior to the inserting (A), concurrently with the inserting (A), or subsequent to the inserting (A).

In some embodiments, the method further includes: (G) attaching, aided at least in part by the light emitted from the one or more light sources, the connection portion of the anvil to the trocar. In some such embodiments, at least one light source in the one or more light sources changes color, changes intensity, flashes or any combination thereof when the connection portion of the anvil is properly attached to the trocar.

In some embodiments, the method further includes (H) drawing the anvil proximally toward the stapler head such that the desired sections of the first and second segments of the tubular organ are adjacent to each other. In some such embodiments, the drawing (H) is achieved by rotating the knob in a second direction opposite to the first direction.

In some embodiments, the method further includes: (I) stapling the desired sections of the first and second segments of the tubular organ together with staples of the stapling assembly; (J) cutting through the desired sections of the first and second segments of the tubular organ adjacent to the staples; and (K) removing the stapler head and anvil of the surgical instrument from the tubular organ.

The instruments and methods of the present disclosure have other features and advantages that will be apparent from, or are set forth in more detail in, the accompanying drawings, which are incorporated herein, and the following Detailed Description, which together explain certain principles of exemplary embodiments of the present disclosure.

### BRIEF DESCRIPTION OF THE DRAWINGS

The accompanying drawings, which are incorporated into and constitute a part of this specification, illustrate one or more exemplary embodiments of the present disclosure and, together with the Detailed Description, explain the principles and implementations of exemplary embodiments of the invention. The accompanying drawings are not necessarily to scale. The specific design features of the present invention as disclosed herein, including, for example, specific dimensions, orientations, locations, and shapes will be determined in part by the intended application and use environment. In addition, the components illustrated in the figures are combinable in any useful number and combination.

In the drawings:

FIG. 1A is a schematic perspective view illustrating an exemplary surgical instrument in accordance with some exemplary embodiments of the present disclosure;

FIG. 1B is a schematic diagram illustrating an exemplary use of the surgical instrument of FIG. 1A in accordance with some exemplary embodiments of the present disclosure;

FIG. 2A is a schematic perspective view illustrating an exemplary stapler head, in which a trocar is in an extended position, in accordance with some exemplary embodiments of the present disclosure;

FIG. 2B is a schematic perspective view illustrating the exemplary stapler head of FIG. 2A, in which a trocar is in a retracted position, in accordance with some exemplary embodiments of the present disclosure;

FIG. 3A is a schematic perspective view illustrating an exemplary stapler head, in which a trocar is in an extended position, in accordance with an alternative exemplary embodiment of the present disclosure;

FIG. 3B is a schematic perspective view illustrating an exemplary stapler head, in which a trocar is in an extended position, in accordance with another alternative exemplary embodiment of the present disclosure;

FIG. 3C is a schematic front view illustrating an exemplary stapler head in accordance with still another alternative exemplary embodiment of the present disclosure;

FIG. 3D is a schematic front view illustrating an exemplary stapler head in accordance with still another alternative exemplary embodiment of the present disclosure;

FIG. 3E is a schematic perspective view illustrating an exemplary stapler head, in which a trocar is in a retracted position, in accordance with still another alternative exemplary embodiment of the present disclosure;

FIG. 3F is a schematic perspective view illustrating an exemplary stapler head, in which a trocar is in a retracted position, in accordance with still another alternative exemplary embodiment of the present disclosure;

FIG. 3G is a schematic perspective view illustrating an exemplary stapler head, in which a trocar is in a retracted position, in accordance with still another alternative exemplary embodiment of the present disclosure;

FIG. 3H is a schematic perspective view illustrating an exemplary stapler head, in which a trocar is in a retracted position, in accordance with still another alternative exemplary embodiment of the present disclosure;

FIG. 4A is a schematic perspective view illustrating an exemplary stapler head and an anvil disengaged from each other in accordance with some exemplary embodiments of the present disclosure;

FIG. 4B is a schematic perspective view illustrating an exemplary stapler head and an anvil engaged from each other in accordance with some exemplary embodiments of the present disclosure;

FIGS. 5A and 5B are flow charts collectively illustrating a method in accordance with some exemplary embodiments of the present disclosure;

FIG. 6 is a schematic perspective view illustrating an exemplary surgical instrument in accordance with some exemplary embodiments of the present disclosure;

FIG. 7 is a schematic perspective view illustrating an exemplary surgical instrument in accordance with some exemplary embodiments of the present disclosure;

FIG. 8A is a schematic view illustrating an exemplary lighting device in accordance with some exemplary embodiments of the present disclosure;

FIGS. 8B and 8C are schematic views illustrating the use of the exemplary lighting device of FIG. 8A in accordance with some exemplary embodiments of the present disclosure;

FIG. 9A is a schematic perspective view illustrating an exemplary stapler head in accordance with some exemplary embodiments of the present disclosure;

FIG. 9B is a schematic exploded view illustrating the exemplary stapler head of FIG. 9A in accordance with some exemplary embodiments of the present disclosure;

FIG. 9C is a schematic perspective view illustrating the exemplary stapler head of FIG. 9A disengaged with an anvil in accordance with some exemplary embodiments of the present disclosure;

FIGS. 9D and 9E are a schematic perspective view and a cross-section view illustrating the exemplary stapler head of FIG. 9A engaged with an anvil in accordance with some exemplary embodiments of the present disclosure;

FIGS. 9F-9H are schematic views illustrating exemplary illumination enhancements produced by the exemplary stapler head of FIG. 9A in accordance with some exemplary embodiments of the present disclosure;

FIGS. 10A and 10B are schematic views illustrating an exemplary illumination enhancement in accordance with some exemplary embodiments of the present disclosure; and

FIGS. 11A and 11B are schematic views illustrating an exemplary illumination enhancement in accordance with some exemplary embodiments of the present disclosure.

### DETAILED DESCRIPTION

The present disclosure provides circular and/or end-to-end anastomosis surgical instruments that use light (e.g., white and/or near infrared light) to better visualize the location of the intended staple line/plane and trocar within tubular organs (e.g., rectum) before the staples are deployed. In various embodiments, the present disclosure employs light in one or more wavelength ranges (e.g., white light and/or NIR wavelengths) disposed at the trocar and/or the stapling assembly (e.g., around the circular staple line) to increase the visibility of the stapler head and to provide improved visual feedback to surgeons. This enables surgeons to correctly position the stapler head of the surgical instrument within tubular organs and accurately deploy the trocar of the stapler head transrectally. Accordingly, this allows for more consistent, reproducible, and standardized procedures to reduce the leak rates in colorectal anastomoses.

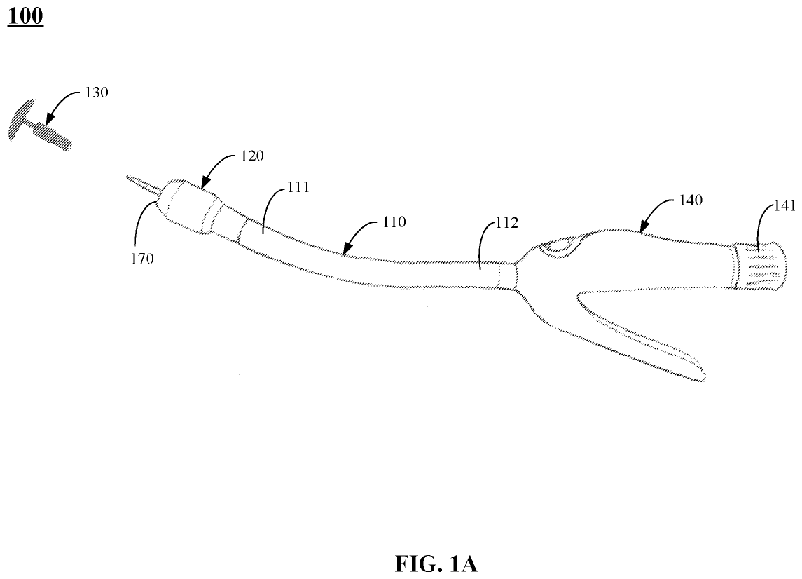

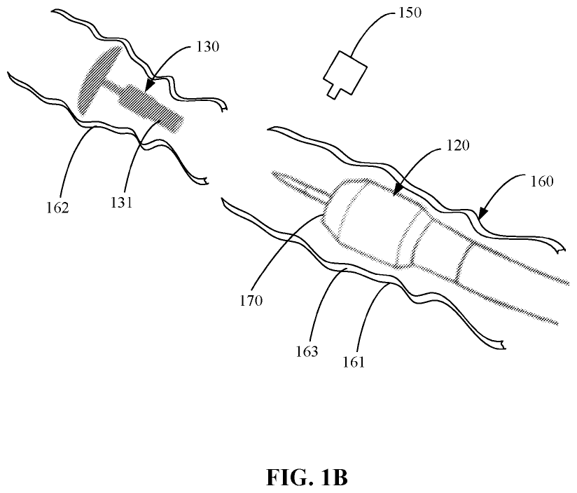

Referring now to the drawings, where like reference numerals indicate like elements throughout, there is shown in FIGS. 1A and 1B an exemplary surgical instrument 100 in accordance with some embodiments of the present disclosure. The surgical instrument 100 is configured for treating tubular organs of patients. Examples of a tubular organ include but are not limited to rectum and colon.

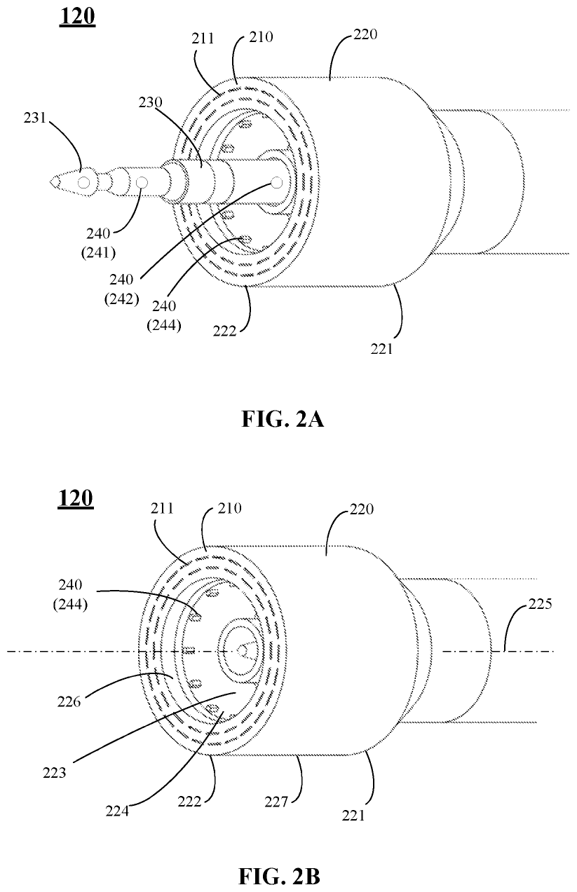

The surgical instrument 100 includes a shaft assembly 110 and a stapler head 120. The shaft assembly 110 has a distal end portion 111 and a proximal end portion 112. The stapler head 120 is disposed at the distal end portion 111 of the shaft assembly 110 and configured to operably support a circular staple cartridge (e.g., the circular staple cartridge 210 illustrated in FIG. 2A). To treat a tubular organ 160 of a patient, the stapler head 120 is to be inserted into a segment 161 of the tubular organ 160 and then engaged with an anvil 130 that has been placed in another segment 162 of the tubular organ 160. Once the stapler head 120 is engaged with the anvil 130, the stapler head 120 can be operated to fire staples (e.g., the staples 211 illustrated in FIG. 2A) to suture together the segment 161 and the segment 162 of the tubular organ 160.

In some embodiments, the stapler head 120 or one or more components of the stapler head 120 may be formed with the distal end portion of the shaft assembly, permanently connected to the distal end portion of the shaft assembly, or removably attached to the distal end portion of the shaft assembly. The stapler head 120 or one or more components of the stapler head 120 may be disposable or non-disposable.

Referring to FIGS. 2A and 2B, in various embodiments, the stapler head 120 includes a stapling assembly 220, a trocar 230, and one or more light sources 240. The stapling assembly 220 is configured for operably supporting a circular staple cartridge 210. The circular staple cartridge 210 may include one, two, or more than two circumferentially spaced and/or staggered rows of staples 211 therein. The stapling assembly 220 may be formed with the distal end portion of the shaft assembly, permanently connected to the distal end portion of the shaft assembly, or removably attached to the distal end portion of the shaft assembly. In embodiments where the stapling assembly 220 is removably attached to the distal end portion of the shaft assembly, the stapling assembly 220 may be disposable or non-disposable.

In some embodiments, the stapling assembly 220 has a circumferential side wall 221 with a distal edge 222 that defines a staple line/plane (e.g., the staple line/plane 170 illustrated in FIGS. 1A and 1B). In some embodiments, the stapling assembly 220 further includes an interior base 223 located proximally away from the distal edge 222 (e.g., on the proximal side with respect to the distal edge 222). Collectively, the circumferential side wall 221 and the interior base 223 forms a recess 224, e.g., the space within the stapling assembly 220 between the distal edge 222 and the interior base 223.

The trocar 230 is configured for puncturing tissues of tubular organs and for engaging with the anvil 130. The trocar 230 is connected to and supported by the stapling assembly 220, the shaft assembly 110 or both of the stapling assembly 220 and the shaft assembly 110. In some embodiments, the trocar 230 is operably movable (e.g., can be operated to move, for instance, through the handle assembly 140) relative to the stapling assembly 220 along an axis 225 of the stapling assembly 220 between an extended position illustrated in FIG. 2A and a retracted position illustrated in FIG. 2B. In some embodiments, the trocar 230 has a distal tip portion 231. When the trocar 230 is in the extended position, the distal tip portion 231 of the trocar 230 is exposed distally relative to the distal edge 222 of the stapling assembly 220, e.g., the distal tip portion 231 of the trocar 230 being positioned at the distal side of the distal edge 222 of the stapling assembly 220. When the trocar 230 is in the retracted position, the distal tip portion 231 of the trocar 230 is concealed proximally relative to the distal edge 222 of the stapling assembly 220, e.g., the distal tip portion 231 of the trocar 230 being positioned at the proximal side of the distal edge 222 of the stapling assembly 220. In some embodiments, when the trocar 230 in the retracted position, the distal tip portion 231 of the trocar 230 is concealed completely within the stapling assembly 220 and/or the shaft assembly 110, e.g., the distal tip portion 231 of the trocar 230 being positioned at the proximal side of the interior base 223 of the stapling assembly 220.

The one or more light sources 240 are disposed at the trocar 230, the stapling assembly 220, or both of the trocar 230 and the stapling assembly 220 to facilitate visualization and precise positioning of the stapler head 120 in tubular organs. For instance, in some embodiments, the one or more light sources 240 are configured to emit light that is able to transmit through a wall of a tubular organ of a patient, such as the wall 163 of the tubular organ 160 illustrated in FIG. 1B. This allows a user of the surgical instrument 100 (e.g., a surgeon) to locate the stapler head 120, including the trocar 230, the distal edge 222 of the stapling assembly 220 or both, in the tubular organ 160 of the patient, for instance, by visualizing and/or detecting light transmitted through the wall 163 of the tubular organ 160. Thus, in colorectal surgeries where an anastomosis is required, the intended staple line/plane 170 (defined by the distal edge 222 of the stapling assembly 220) and the position of the trocar 230 are visible to the surgeon. With the direct line of sight to the stapler assembly, the surgeon can place the stapler assembly correctly and precisely in the tubular organ to avoid imprecise placement that could lead to anastomotic leaks and/or unnecessary additional resections of bowel, as well as increased operative time and increased stress for the surgeon.

The one or more light sources 240 can be of various types, including but not limited to light-emitting diodes (LEDs), fiber optics and fluorescence-embedded polymers. Individual light sources can be of the same type or different types. For instance, in an embodiment, each of the one or more light sources 240 is an LED. In another embodiment, each of the one or more light sources 240 is an optic fiber. In a further embodiment, each of the one or more light sources 240 is made of a fluorescent-embedded material such as a polymer containing a near infrared fluorescent material. In some embodiments, the one or more light sources 240 include any combination of at least one optic fiber, at least one LED, and/or at least one light source made of a fluorescent-embedded material.

The one or more light sources 240 or any individual light source in the one or more light sources 240 may be operated to emit light at any desired range. The desired range of light may be wide or narrow. It may be a single continuous range or a combination of multiple ranges. For instance, the desired range of light may include a near-infrared light range, a visible light range (e.g., white light range), an ultraviolet light range, one or more targeted narrow-bands, or any combination thereof. As used herein, the near-infrared light range refers to the light range of about 600 nm to about 1300 nm. In some cases, the near-infrared light range refers to the light range of about 600 nm to about 1100 nm or about 700 nm to about 1300 nm. The visible light range refers to the light range of about 400 nm-about 700 nm. In some cases, the visible light range refers to the light range of about 400 nm-about 650 nm. The ultraviolet light range refers to the light range of about 10 nm to about 400 nm.

In some embodiments, the one or more light sources 240 may emit light of a variety of peak wavelengths to increase visibility and communication. A light source emitting light of a different peak wavelength than other light sources may be disposed at the trocar to make its appearance easily distinguishable to a user and/or a camera. The one or more light sources 240 may also configured to provide dynamic cues, such as flashing at specific intervals and/or changing in intensity and/or wavelength, to communicate additional information to the user and/or camera.

In some embodiments, at least one light source in the one or more light sources 240 emits light in the near-infrared light range. Near infrared light has inherent transmissivity through biological tissues. All biological tissues are composite structures that can absorb specific wavelengths of light. The chemical and physical properties of different molecules in biologic tissue affect the amount and wavelengths of light that can be absorbed by the tissue. In the visible light spectrum, intense light absorption of the hemoglobin molecule and light loss due to scattering prevents transmission of visible light more than a few millimeters of tissue. The entire near infrared spectrum is defined as light generally having wavelengths up to about 2500 nm. In the infrared spectrum above 1300 nm, water present in tissue absorbs a large amount of infrared light, thus limiting infrared light transmission through tissue to a short distance. In the near infrared range of 700 to 1300 nm, a significant portion of near infrared light can be transmitted through several centimeters of biological tissue. This window of high transmissivity is caused by the absence of molecules that absorb near infrared light between 700 nm and 1300 nm. Embodiments of the present disclosure capitalize on the fact that this spectrum of near infrared energy can be transmitted through several centimeters of biological tissue and allow for transillumination of an organ during a surgical procedure.

In some embodiments, at least one light source in the one or more light sources 240 is operable to emit light in any one of a plurality of wavelength ranges (e.g., a visible light range, a near infrared light range, and/or an ultraviolet light range). In some such embodiments, the plurality of wavelength ranges includes a first wavelength range (e.g., a visible light range) and a second wavelength range (e.g., a near infrared light range) that is different from the first wavelength range. In some embodiments, the first wavelength range is in a white light range and the second wavelength range is in a near-infrared light range.

In some embodiments, at least one light source in the one or more light sources 240 is operable to alter its state, e.g., by flashing, changing color, changing intensity or the like. For instance, in some embodiments, when the anvil 130 is properly aligned with the trocar 230 or properly attached to the trocar 230, at least one light source in the one or more light sources 240 can be operated to alter its state, such as by flashing and/or changing color. This indicates to the surgeon the proper alignment or attachment of the anvil 130 to the trocar 230, and that the anvil 130 and trocar 230 are ready to close. In some embodiments, a sensor and/or a switch is used to detect the alignment or attachment of the anvil 130 to the trocar 230. Examples of such a sensor and/or switch include but are not limited to an optical sensor, capacitive switch or mechanical switch such as a membrane switch or snap dome switch.

In some embodiments, the light transmitted through the wall 163 of the tubular organ 160 of the patient is detected by a camera 150 positioned outside of the tubular organ 160 of the patient as illustrated in FIG. 1B. The camera 150 may be a component of a laparoscope or endoscope. In some embodiments, the light detected by the camera 150 is displayed (e.g., on a screen, a display or the like) to provide real-time visualization of where the trocar, the distal edge of the stapling assembly or both are located in the tubular organ of the patient.

The one or more light sources 240 can include any suitable number of light sources and can be disposed at any suitable locations. For instance, in some embodiments, the one or more light sources 240 include at least one light source disposed at the distal tip portion 231 of the trocar 230 (referred herein as the at least one first light source and designated “241”) , at least one light source disposed at a proximal base portion of the trocar 230 (referred herein as the at least second light source and designated “242”), at least one light source disposed on the interior base 223 of the stapling assembly (referred herein as the at least third light source and designated “243”), at least one fourth light source disposed on an inner surface 226 of the circumferential side wall of the stapling assembly (referred herein as the at least fourth light source and designated “244”), at least one fifth light source disposed on an outer surface 227 of the circumferential all of the stapling assembly (referred herein as the at least fifth light source and designated “245”), or any combination thereof. The one or more light sources 240 increase visualization of the stapler head, in particular, the trocar and the circular staple line, and enable surgeons to correctly position the stapler head 120 within tubular organs (e.g., rectum) and accurately deploy the trocar 230 of the stapler head 120 transrectally. If light sources are located on the outside of a stapler head, the light sources may be positioned relative to the outer plane of the stapler head (e.g., the plane corresponding to the distal edge 222) so as to optimize their visibility for a surgical camera (e.g., a camera positioned outside of the operation field).

As a non-limiting example, FIGS. 2A and 2B illustrate that the one or more light sources 240 include a plurality of first light sources 241, a single second light source 242 and a plurality of fourth light sources 244. The first light sources 241 are disposed at the distal tip portion 231 of the trocar 230. The first light sources 241 may or may not be uniformly spaced along the trocar. In some embodiments, the first light sources 241 are spaced apart along the trocar with known intervals, and may serve as markers for detection by a marker-based augmented reality system. The single second light source 242 is disposed at the proximal base portion of the trocar 230. The fourth light sources 244 are disposed on an inner surface 226 of the circumferential side wall 221 of the stapling assembly 220. The distribution of the fourth light sources can be either uniformly or non-uniformly. In some embodiments, the fourth light sources 244 are disposed circumferentially around the axis 225 of the stapling assembly on the inner surface of the circumferential side wall of the stapling assembly. In some such embodiments, the fourth light sources 244 are distributed around the axis of the stapling assembly with known angular intervals. In some embodiments, at least one fourth light in the plurality of fourth light sources 244 is elongated along the axis of the stapling assembly. In some embodiments, each of the plurality of fourth light sources 244 is elongated along the axis of the stapling assembly.

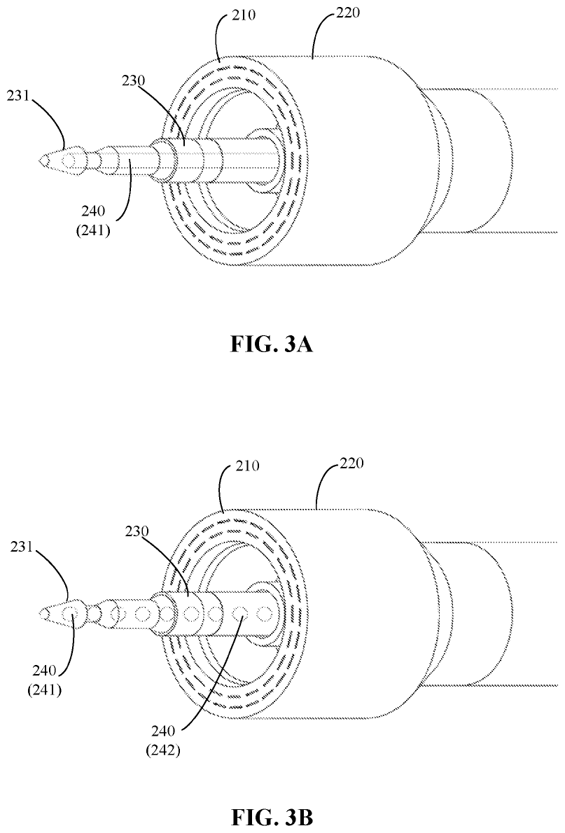

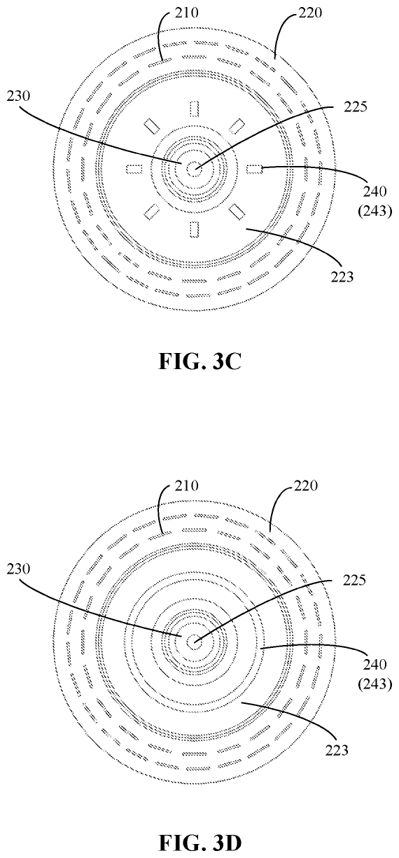

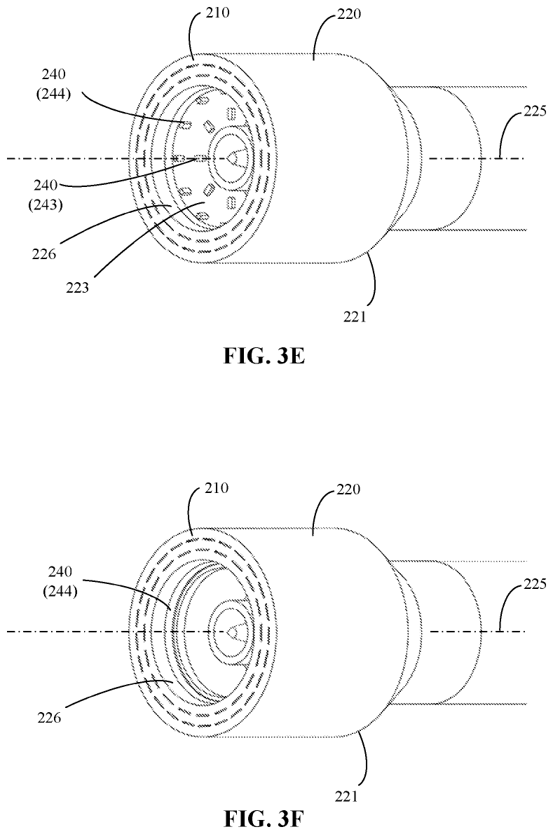

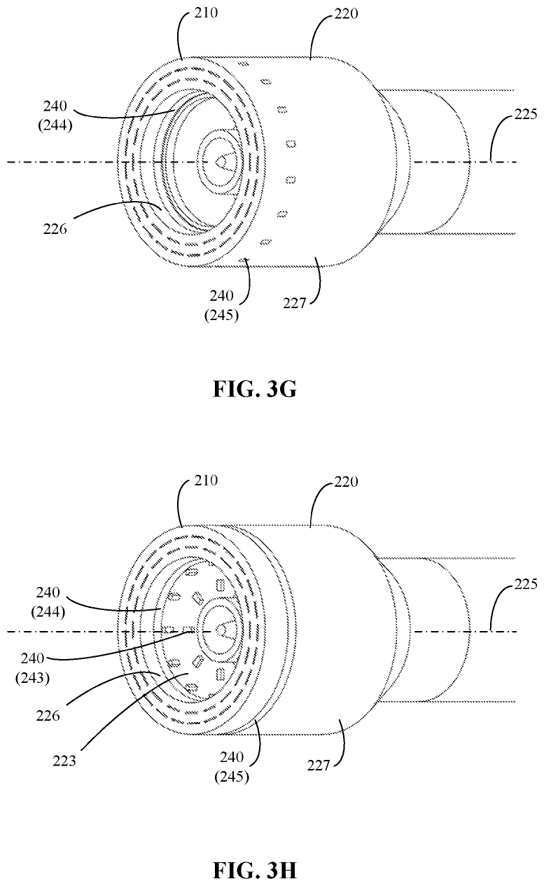

As another non-limiting example, FIG. 3A illustrates that the one or more light sources 240 include a single first light source 241. The single first light source 241 may be elongated along at least a portion of the trocar 230. The elongated first light source may be an optic fiber, and/or made of a fluorescent-embedded material. In some embodiments, the elongated light source may extend to the proximal base portion of the trocar and thus serve as both of the first and second light sources. In some embodiments, the distal tip portion 231 of the trocar 230 or a substantial portion (e.g., at least 50%) of the trocar 230 is made of a fluorescent-embedded material and serves as the first and/or second light source.

As still another non-limiting example, FIG. 3B illustrates that the one or more light sources 240 include a plurality of first light sources 241 and a plurality of second light sources 242. Collectively, the plurality of first light sources 241 and the plurality of second light sources 242 are disposed spatially over a substantial portion (e.g., at least 50%) of the trocar 230. The first and second light sources may be uniformly or non-uniformly distributed along the trocar. In some embodiments, the first and/or second light sources are distributed along the trocar with known intervals, and may serve as markers for detection by a marker-based augmented reality system.

As still another non-limiting example, FIG. 3C illustrates that the one or more light sources 240 include a plurality of third light sources 243 disposed circumferentially around the axis 225 of the stapling assembly on the interior base 223 of the stapling assembly. The third light sources 243 may be uniformly or non-uniformly distributed on the interior base 223 of the stapling assembly, and may have any suitable shape and size. In some embodiments, each of the plurality of third light sources 243 is elongated radially with respect to the axis 225 of the stapling assembly.

As still another non-limiting example, FIG. 3D illustrates that the one or more light sources 240 include a single third light source 243. The single third light source 243 may have any suitable shape and size, and may or may not encircle the axis 225 of the stapling assembly. In some embodiments, the single third light source 243 has a ring shape that encircles the axis 225 of the stapling assembly.

As still another non-limiting example, FIG. 3E illustrates that the one or more light sources 240 include a plurality of third light source 243 and a plurality of fourth light sources 244. The third light sources 243 are disposed circumferentially around the axis 225 of the stapling assembly on the interior base 223 of the stapling assembly 220, and the fourth light sources 244 are disposed on the inner surface 226 of the circumferential side wall 221 of the stapling assembly 220.

As still another non-limiting example, FIG. 3F illustrates that the one or more light sources 240 include a single fourth light source 244 disposed on the inner surface 226 of the circumferential side wall 221 of the stapling assembly 220. The single fourth light source 244 may have any suitable shape and size, and may or may not encircle the axis 225 of the stapling assembly. In some embodiments, the single fourth light source 244 has a ring shape that encircles the axis 225 of the stapling assembly.

As still another non-limiting example, FIG. 3G illustrates that the one or more light sources 240 include a single fourth light source 244 and a plurality of fifth light sources 245. The single fourth light source 244 is disposed on the inner surface 226 of the circumferential side wall 221 of the stapling assembly 220. The fifth light sources 245 are disposed, uniformly or non-uniformly, on the outer surface 227 of the circumferential side wall 221 of the stapling assembly 220. In some embodiments, the fourth light sources 244 are disposed circumferentially around the axis 225 of the stapling assembly on the outer surface of the circumferential side wall of the stapling assembly. In some embodiments, the fifth light sources 245 are distributed around the axis 225 of the stapling assembly with known angular intervals, and may serve as markers for detection by a marker-based augmented reality system. In some embodiments, at least one fifth light source in the plurality of fifth light sources 245 is elongated along the axis 225 of the stapling assembly. In some embodiments, each of the plurality of fifth light sources 245 is elongated along the axis 225 of the stapling assembly.

As still another non-limiting example, FIG. 3H illustrates that the one or more light sources 240 include a plurality of third light source 243 disposed circumferentially around the axis 225 of the stapling assembly 220 on the interior base 223 of the stapling assembly 220. The one or more sources 240 also includes a plurality of fourth light sources 244 disposed on the inner surface 226 of the circumferential side wall 221 of the stapling assembly 220. The one or more light sources 240 further include a single fifth light source 245 disposed on the outer surface 227 of the circumferential side wall 221 of the stapling assembly 220. The single fifth light source 245 may have any suitable shape and size, and may or may not encircle the axis 225 of the stapling assembly. In some embodiments, the single fifth light source 245 has a ring shape that encircles the axis 225 of the stapling assembly.

The single fifth light source 245 may be a standalone component. In some embodiments, the single fifth light source 245 may be adapted to fix on an existing device having a component similar to the stapling assembly 220 to enable visualization and positioning of the existing device. The single fifth light source 245 may be a substance embedded with one or more light sources that can be connected to a power source. This would allow for the existing device (e.g., a non-lighted) circular stapler to have a basic light guiding element.

While FIGS. 2A-3H illustrate the components with particular shapes, it should be noted that they are by way of examples and the present invention is not limited thereof. The stapler head of the present disclosure can be configured with additional, optional and/or alternative components that may have different shapes and/or sizes and may be arranged at different positions. The first, second, third, fourth, fifth or other additional/optional/alternative light sources can be implemented and operated independently of each other, or combined with each other in any suitable manner. For instance, the first light source(s) can be implemented and operated independently of the second, third, fourth, fifth and/or other additional/optional/alternative light sources. The first light source(s) can also be implemented and operated along with the second, third, fourth, fifth and/or other additional/optional/alternative light sources. Similarly, the second light source(s) can be implemented and operated independently of or along with the first, third, fourth, fifth or other additional/optional/alternative light sources. The third light source(s) can be implemented and operated independently of or along with the first, second, fourth, fifth or other additional/optional/alternative light sources. The fourth light source(s) can be implemented and operated independently of or along with the first, second, third, fifth or other additional/optional/alternative light sources. The fifth light source(s) can be implemented and operated independently of or along with the first, second, third, fourth or other additional/optional/alternative light sources.

Moreover, the at least one first light source, the at least one second light source, the at least one third light source, the at least one fourth light source, and the at least one fifth light source each can independently consist of a single first/second/third/fourth/fifth light source or include a plurality of first/second/third/fourth/fifth light sources. The first, second, third, fourth, and/or fifth light sources can have any suitable shapes and sizes, including but not limited to those illustrated in the figures. The first, second, third, fourth, and/or fifth light sources can emit light of any desired ranges.

Further, a light source may be identical to another light source in type, shape, size, color, or the like. For instance, identical LEDs may be used for both a first light source and the other light source(s). A light source may also differ from another light source in type, shape, size, color, or the like. For instance, a first light source may be an LED whereas the other light source(s) may be an optic fiber and/or a fluorescence-embedded polymer. Alternatively, a first light source may be an optic fiber or fluorescence-embedded polymer whereas the other light source(s) may be an LED. A first light source may be an LED of a color while the other light source(s) may be an LED of a different color.

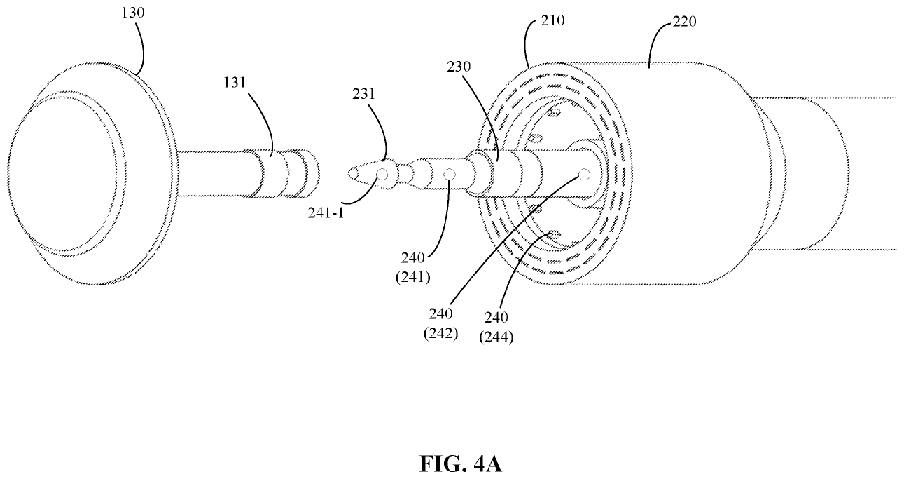

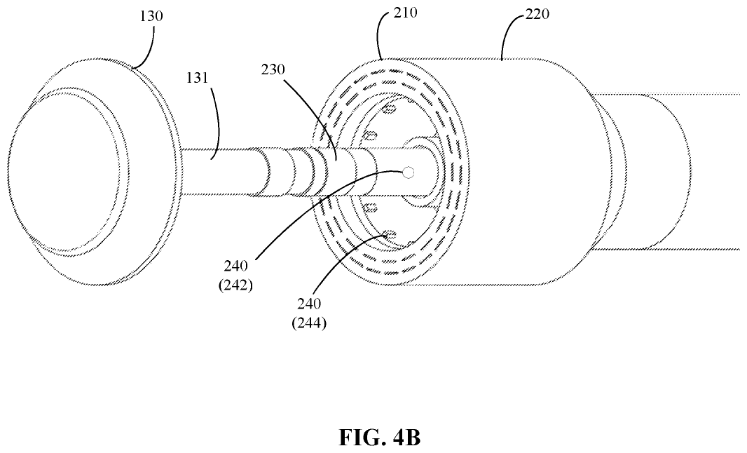

Referring to FIGS. 4A and 4B, in some embodiments, the at least one first light source 241 includes a first light source (e.g., the first light source 241-1) disposed at a very tip of the trocar 230. The first light source at the very tip of the trocar 230 is fully concealed by a complete attachment of the anvil 130 to the trocar 230. While a plurality of first light sources 241 is illustrated, it should be noted that this is by way of example. The present disclosure is not limited thereto. The at least one first light source 241 can include a single first light source, which can be either elongated as illustrated in FIG. 3A or not elongated. In embodiments where the at least one first light source 241 includes a single elongated first light source, at least a portion of the single elongated first light source disposed at the very tip of the trocar 230 will be fully concealed by the complete attachment of the anvil 130 to the trocar 230.

Typically, the at least one first light source 241, the at least one second light source 242, or both are disposed inside of the trocar 230. In embodiments where the at least one first light source 241 is implemented, the trocar 230 is generally made of a material transparent or translucent to the light emitted by the at least one first light source 241. In embodiments where the at least one second light source 242, the trocar 230 is generally made of a material transparent or translucent to the light emitted by the at least one second light source 242. In embodiments where the at least one first and second light sources are implemented, the trocar 230 is generally made of a material transparent or translucent to the light emitted by the at least one first light source 241 and the light emitted by the at least one second light source 242. In some embodiments, the trocar 230 is made of a medical grade plastic. Examples of such a medical grade plastic include but are not limited to acrylic (PMMA), transparent sulfone polymers, polymethyl methacrylate, PVC, polycarbonate, polyethylene terephthalate, polypropylene, polyethylene, styrene methyl methacrylate, and ionomer resin.

Referring back to FIG. 1A, in some embodiments, the stapler head 120 may be operably connected to a handle assembly 140 disposed at the proximal end portion 112 of the shaft assembly 110. In some such embodiments, the operation of the stapler head 120 may be performed through the handle assembly 140. For instance, in some embodiments, the operating of the stapling assembly 220, the trocar 230, the one or more light sources 240, or any combination thereof may be performed through the handle assembly 140.

The operation of the stapler head 120 performed through the handle assembly 140 may be manual, powered or automatic. For instance, in some embodiments, the handle assembly 140 includes a battery to power the one or more light sources 240, to aid the operation of the stapling assembly 220, to aid the operation of the trocar 230, or any combination thereof. In some embodiments, the handle assembly 140 is electrically connected to a power outlet to power the one or more light sources 240, to aid the operation of the stapling assembly 220, to aid the operation of the trocar 230, or any combination thereof. In some embodiments, the handle assembly 140 includes a rotatable knob 141 connected to the trocar 230 for extending or retracting the trocar 230.

In some embodiments, the positioning of the shaft assembly 110 is through the manipulation of the handle assembly 140. In some embodiments, the one or more sources 240 include at least one LED light source. In some such embodiments, a battery may be housed within the handle assembly 140 to power the LED light source(s) through electrical wire(s) contained in the shaft assembly 110. In some embodiments, the one or more sources 240 include at least fiber optic light source. In some such embodiments, a plug for an external light box power source may be attach to the handle assembly to power the fiber optic light source(s) through fiber optic wire(s) contained in the shaft assembly 110. In some embodiments, the one or more sources 240 include both LED and fiber optic light sources. In some such embodiments, a battery may be housed within and/or a plug may be attached to the handle assembly 140 to power both LED and fiber optic light sources.

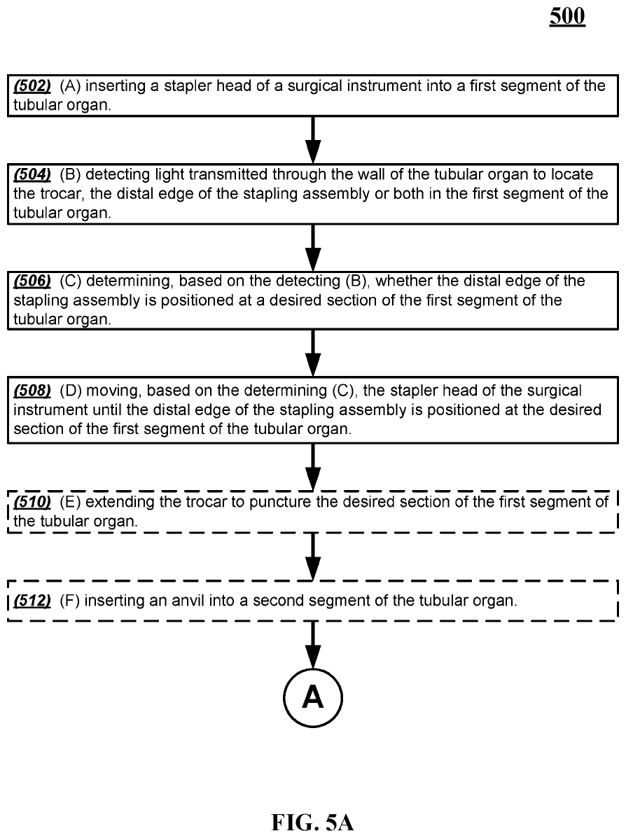

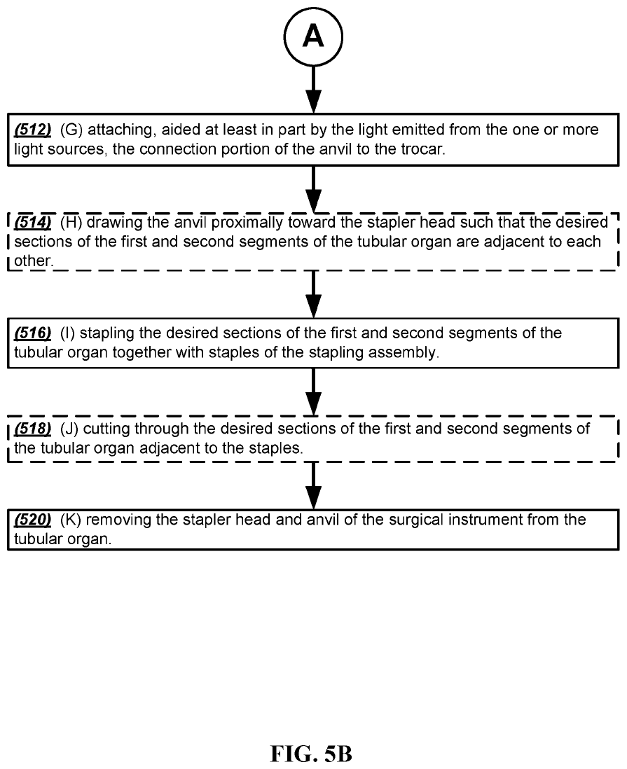

Referring to FIGS. 5A and 5B, there is shown a flowchart illustrating an exemplary method 500 for treating a tubular organ of a patient in accordance with some embodiments of the present disclosure. In the flowchart, the preferred parts of the method are shown in solid line boxes, whereas additional, optional, and/or alternative parts of the method are shown in dashed line boxes. It should be noted that the processes disclosed herein and exemplified in the flowchart can be, but do not have to be, executed in full or in the order as they are presented.

Referring to block 502, in some embodiments, the method 500 includes (A) inserting a stapler head of a surgical instrument into a first segment of the tubular organ. For instance, in some embodiments, the method 500 uses the surgical instrument 100 of the present disclosure to treat a tubular organ 160 of a patient. The method 500 include inserting the stapler head 120 of the surgical instrument 100 into the first segment 161 of the tubular organ 160 as illustrated in FIG. 1B. The stapler head 120 of the surgical instrument 100 includes a stapling assembly 220, a trocar 230 and one or more light sources 240. The stapling assembly 220 has a distal edge 222 to define a staple line/plane 170. The trocar 230 is operably movable relative to the stapling assembly 220 along an axis 225 of the stapling assembly 220. The one or more light sources 240 are disposed at the trocar 230 and/or the stapling assembly 220 to emit light capable of transmitting through a wall 163 of the tubular organ 160.

Referring to block 504, in some embodiments, the method 500 includes (B) detecting light transmitted through the wall of the tubular organ to locate the trocar, the distal edge of the stapling assembly or both in the first segment of the tubular organ. The detecting (B) may include visualization by the user (e.g., a surgeon) of the surgical instrument 100, detection by a camera 150, or both. The camera 150 may be a laparoscopic, endoscopic, and/or robotic camera. The camera 150 may be operable in a single mode, or selectively operable in one of multiple modes. For instance, in some embodiments, the camera 150 may be operable in a white light mode, a near-infrared mode, or both. In some embodiments, the light detected in the detecting (B) is displayed (e.g., on a screen, a display or the like) to provide real-time visualization of where the trocar, the distal edge of the stapling assembly or both are located in the tubular organ of the patient.

Referring to block 506 and block 508, in some embodiments, the method 500 includes (C) determining, based on the detecting (B), whether the distal edge of the stapling assembly is positioned at a desired section of the first segment of the tubular organ. In some embodiments, the method 500 includes (D) moving, based on the determining (C), the stapler head of the surgical instrument until the distal edge of the stapling assembly is positioned at the desired section of the first segment of the tubular organ.

Referring to block 510, in some embodiments, the method 500 includes (E) extending the trocar to puncture the desired section of the first segment of the tubular organ. In some embodiments, the extending (E) is achieved by rotating a knob 141 at a handle assembly 140 of the surgical instrument 100 in a first direction (e.g., clock-wise or counter-clock-wise).

Referring to block 512, in some embodiments, the method 500 includes (F) inserting an anvil into a second segment of the tubular organ. For instance, in some embodiments, the method 500 includes inserting the anvil 130 of the present disclosure into the second segment 162 of the tubular organ as illustrated in FIG. 1B. In some embodiments, the anvil 130 includes a connection portion 131 that can protrude proximally out of a desired section of the second segment of the tubular organ. The inserting (F) can be performed prior to, concurrently with, or subsequently to the inserting (A).

Referring to block 512, in some embodiments, the method 500 includes (G) attaching, aided at least in part by the light emitted from the one or more light sources, the connection portion of the anvil to the trocar. In some embodiments, at least one light source in the one or more light sources alters its state when the connection portion 131 of the anvil is properly attached to the trocar. In some such embodiments, the at least one light source alters its state by changing color, changing intensity, flashing or any combination thereof. This indicates to the surgeon the proper alignment or attachment of the anvil 130 to the trocar 230, and that the anvil 130 and trocar 230 are ready to close. In some embodiments, whether the connection portion 131 of the anvil is properly attached to the trocar is detected by a sensor or a switch.

Referring to block 514, in some embodiments, the method 500 includes (H) drawing the anvil proximally toward the stapler head 120 such that the desired sections of the first and second segments of the tubular organ are adjacent to each other. In some embodiments, the drawing (H) is achieved by rotating the knob 141 in a second direction opposite to the first direction. In some embodiments, the drawing (H) is dependent on the tissue thicknesses of the tubular organ.

Referring to block 516, in some embodiments, the method 500 includes (I) stapling the desired sections of the first and second segments of the tubular organ together with staples of the stapling assembly. For instance, in some embodiments, the method includes firing the staples 211 within the stapling assembly 220 using the handle assembly 140 to staple the first desired sections of the first segment 161 and the second segment 162 of the tubular organ 160.

Referring to block 518 and block 520, in some embodiments, the method 500 includes (J) cutting through the desired sections of the first and second segments of the tubular organ adjacent to the staples and (K) removing the stapler head and anvil of the surgical instrument from the tubular organ. In some embodiments, care is taken to ensure that the desired sections of the first and second segments of the tubular organ are properly stapled together before the cutting (J) and the removing (K).

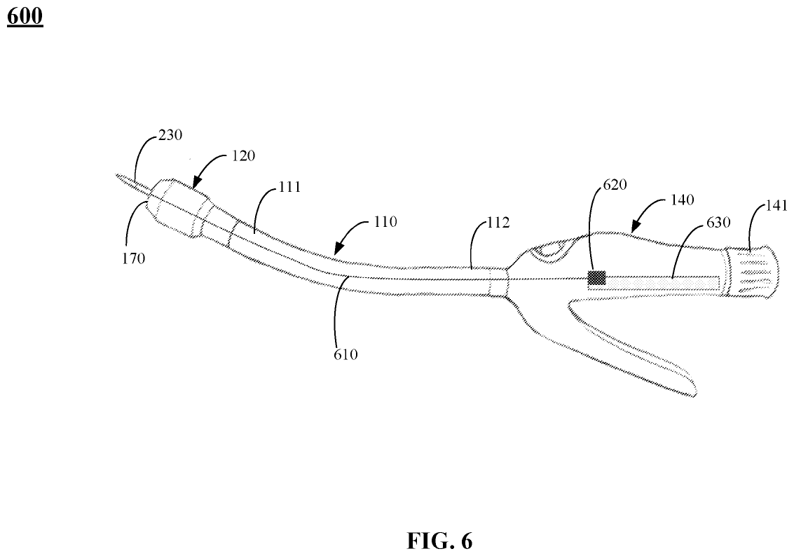

FIG. 6 illustrated an exemplary surgical instrument 600 in accordance with some embodiments of the present invention. The surgical instrument 600 is similar to the surgical instrument 100 disclosed herein. For instance, in some embodiments, the surgical instrument 600 includes the shaft assembly 110 having the distal end portion 111 and the proximal end portion 112. In some embodiments, the surgical instrument 600 also includes the stapler head 120 disposed at the distal end portion 111 of the shaft assembly 110 and configured to engage with the anvil 130. In some embodiments, the stapler head 120 is operably connected to the handle assembly 140 disposed at the proximal end portion 112 of the shaft assembly 110.

In some embodiments, the surgical instrument 600 includes one or more optical components 610 for transmitting light from the handle assembly 140 through the shaft assembly 110 to the stapler head 120 for illuminating the trocar 230 and/or operation working site. An optical component 610 may be an optical fiber, a fiber optic cable, a light pipe or the like. An optical fiber may be made of glass or plastic, and is typically flexible. A fiber optic cable may contain a varying number of glass fibers, from a few up to a couple hundred. A light pipe may be made of metal or plastic, and can be rigid (e.g., solid transparent plastic rod) or flexible (e.g., similar to an optical fiber). Light pipes may also be referred to as homogenizing rods, light guides, homogenizers, or light funnels.

In an exemplary embodiment, a single optiocal component (e.g., a single optical fiber or a single light pipe) is used to transmit the light. In some embodiments, a plurality of optical components is used to transmit the light. In such embodiments, the multiple optical components may be bundled with each other or separated from each other. In some embodiments, the distal end(s) of the optical component(s) 610 may be arranged so as to create a desired pattern of light delivered to the stapler head 120 and/or around the trocar 230. Non-limiting examples of a desired pattern include but are not limited to a circle, crosshair, or the like. In some embodiments, the distal end(s) of the optical component(s) 610 may be arranged on the interior base 223 of the stapling assembly 220 to create a pattern of light similar to those light sources 240 (243) illustrated in FIGS. 3C and 3D. However, the present invention is not limited thereto. The distal end(s) of the optical component(s) 610 may be arranged at other location(s) and/or to create other desired patterns of light.

The optical component(s) 610 may be connected to an internal light source 620 powered by an internal power source 630, which may be housed in the handle assembly 140 and may be removable from the handle assembly 140. The internal power source 630 may include a rechargeable battery or a rechargeable battery pack.

The optical component(s) 610 may run longitudinally along at least a portion of the length of the shaft assembly 110. The optical component(s) 610 may be held by optic connectors, clips, ferrules, sleeves, optic cable assemblies, or the like. Additionally or alternatively, the optical component(s) 610 may be guided by channels cut into existing components of the shaft assembly 110 and/or of the stapler head 120, such as the component for advancing and retracting the trocar 230.

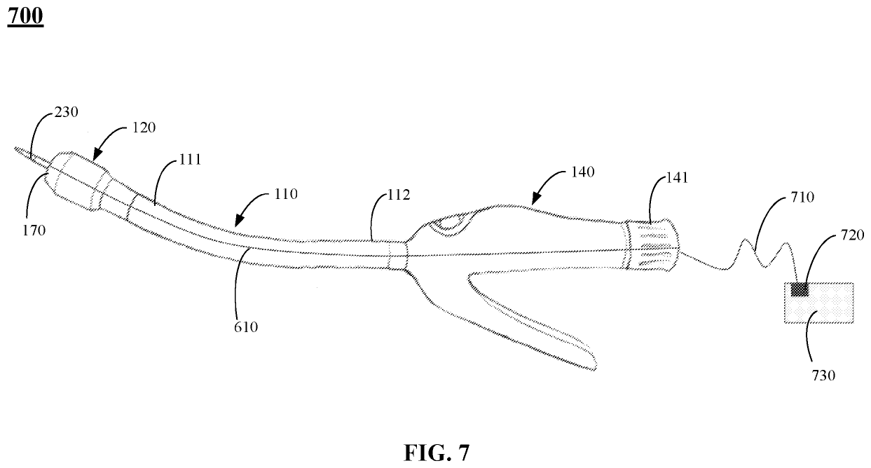

FIG. 7 illustrates an exemplary surgical instrument 700 in accordance with some embodiments of the present invention. The surgical instrument 700 is substantially the same as the surgical instrument 600 disclosed herein, except the optical component(s) 610 (e.g., optical fiber or light pipe) in the exemplary surgical instrument 700 is connected to an external light source 720 (e.g., not housed in the handle assembly 140). A cable or wire 710 or the like may be used to connect the optical component(s) 610 in the exemplary surgical instrument 700 with the external light source 720. In some embodiments, the external light source 720 is powered by an external power source 730, which may include a rechargeable battery, a rechargeable battery pack, and/or a utility power source.

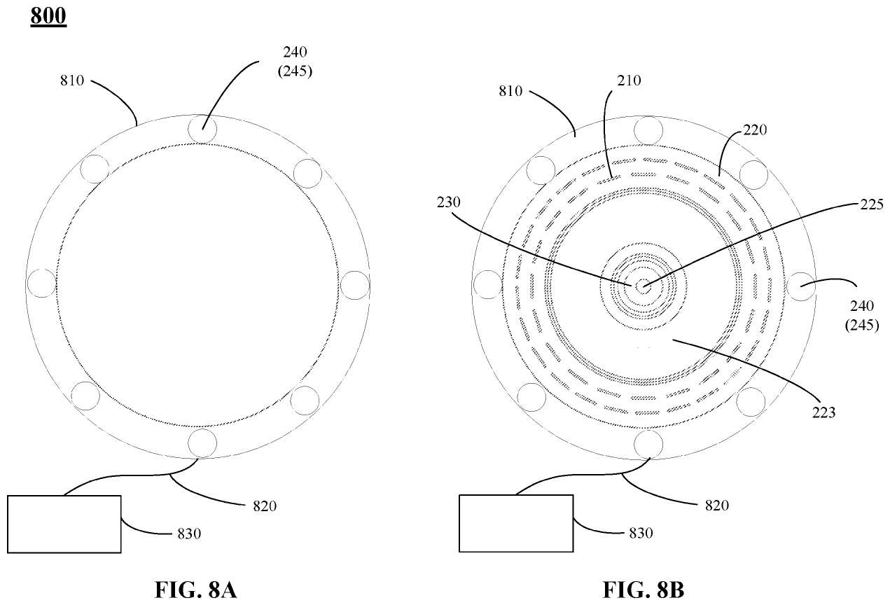

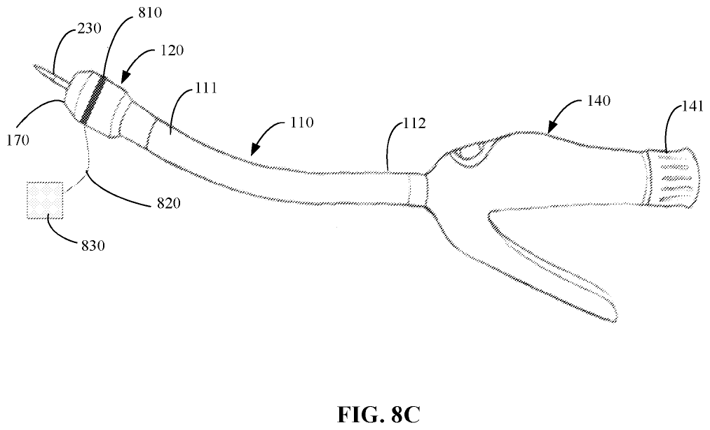

FIG. 8A illustrates an exemplary lighting device 800 in accordance with some embodiments of the present invention. The lighting device 800 includes a ring 810 configured to fit around a surgical instrument. While FIGS. 8B and 8C illustrate the ring 810 fitted around the stapler head 120 of a surgical instrument disclosed herein, it should be noted that this is by way of example and is non-limiting. The ring 810 of the lighting device can be configured to fit around a surgical instrument disclosed herein and/or any other commercially available surgical instrument (e.g., commercially available staplers).

In some embodiments, the ring 810 is flexible (e.g., deformable, stretchable, bendable or the like). In some embodiments, the ring 810 is made of a material that is biocompatible, flexible, and durable. Non-limiting examples of such a material include nut are not limited to silicone, thermoplastic elastomers (TPE), and polyurethane. In some embodiments, the ring 810 is configured to fit around a stapler head of a surgical instrument (which can be a surgical instrument disclosed herein and/or other commercially available surgical instrument) that has an outer diameter ranging from about 16 millimeters (mm) to about 34 mm. In an exemplary embodiment, the ring 810 is configured to fit around a stapler head having an outer diameter ranging from about 15 mm to about 25 mm, about 20 mm to about 30 mm, or about 25 mm to about 35 mm.

The lighting device 800 also includes one or more light sources, such as the light source 240 (245), embedded in the ring 810. In an exemplary embodiment, the lighting device 800 includes a single light source (e.g., a single LED). In some embodiments, the lighting device 800 includes a plurality of light sources distributed along the ring 810 to enhance and/or maximize the visibility. The distribution of the light sources may be uniformly or non-uniformly around the ring 810. In some embodiments, the lighting device 800 includes at least 5, at least 6, at least 7, at least 8, at least 9, or at least 10 light sources. In an exemplary embodiment, the lighting device 800 includes up to 12 light sources.

In some embodiments, the ring 810 (or the light sources embedded in the ring) is connected to a power source 830, for instance, through a wire or cable 820. The power source 830 is configured to power the light source(s) mounted on or embedded in the ring 810. The power source 830 may be internal (e.g., housed in a handle assembly), external (e.g., outside of a handle assembly), or removable. When the ring 810 fits around a stapler head (e.g., the stapler head 120 illustrated in FIG. 8C) and the power source 830 is on, the light source(s) mounted on or embedded in the ring 810 will emit light that illuminates a trocar of the stapler head (e.g., the trocar 230 of the stapler head 120 illustrated in FIG. 8C) and/or adjacent area (e.g., a working site during operation).

In some embodiments, the lighting device 800 includes one or more features configured to fix the ring 810 to an exterior of a stapler head (e.g., the stapler head 120 illustrated in FIG. 8C) and prevent it from accidentally coming off during the operation. The one or more features may include a frictional surface formed on the ring 810 and/or the stapler head, an elasticity of the ring 810, an interlocking mechanism between the ring 810 and the stapler head, an adhesive, a tensioning device, an external support, or any combination thereof. The frictional surface may be a textured and/or rough surface that increase friction of the ring 810 with the stapler head. The elasticity may be achieved and/or improved by selecting a proper elastic material for the ring 810, thereby allowing it to stretch slightly to fit snugly around the stapler head. The interlocking mechanism may be implemented by forming protrusions and/or grooves on the ring 810 to grip the stapler head. The adhesive may be a non-toxic, medical-grade adhesive that can be applied to the inner surface of the ring 810 to help adhere it to the stapler head. The tensioning device may be a screw, a spring, a bias or the like, which can be used to apply tension to the ring 810 against the stapler head, thereby retaining the ring 810 in place. The external support may be a strap, a clip or the like, that can be used to hold the ring 810 in place on the stapler head.

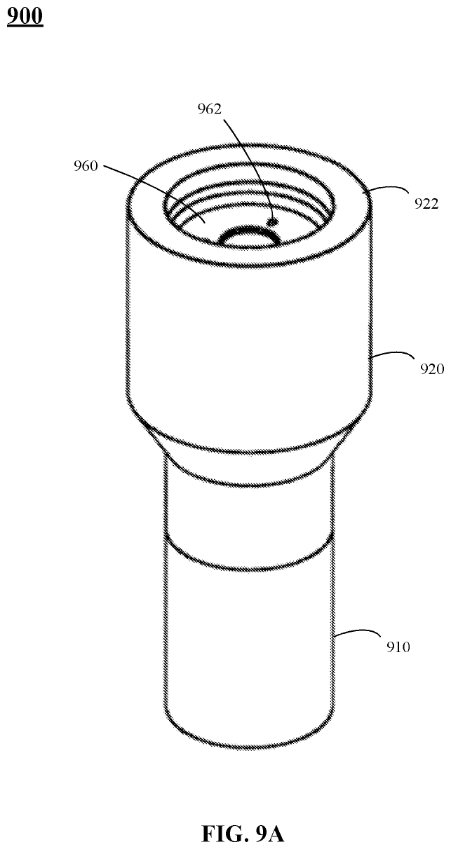

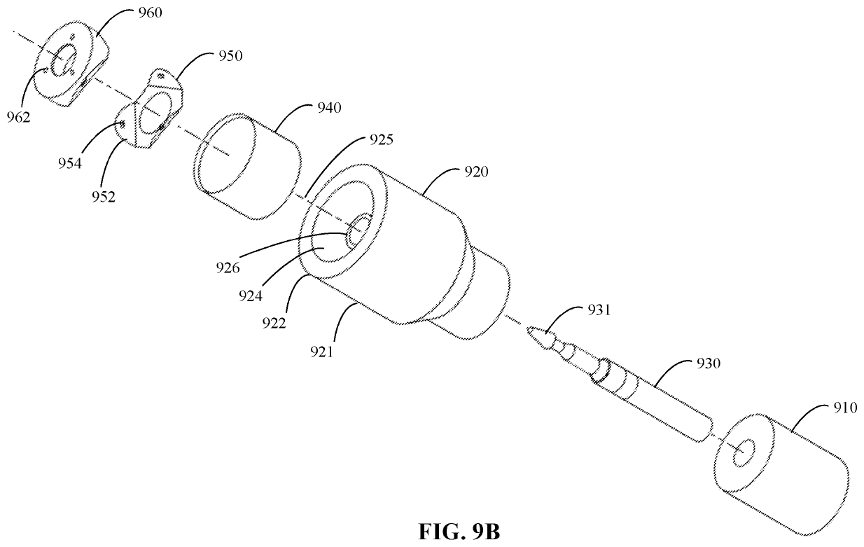

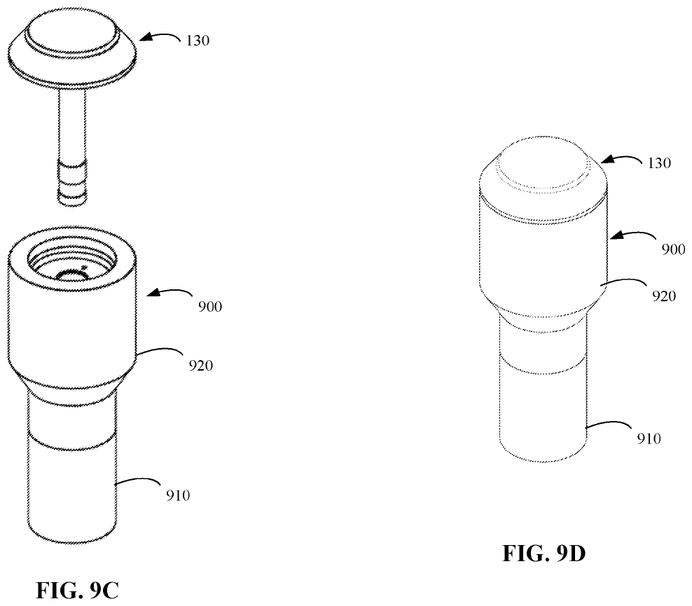

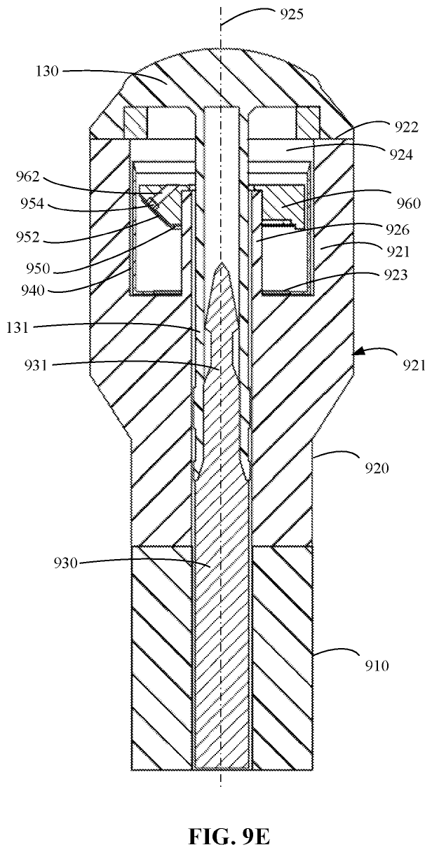

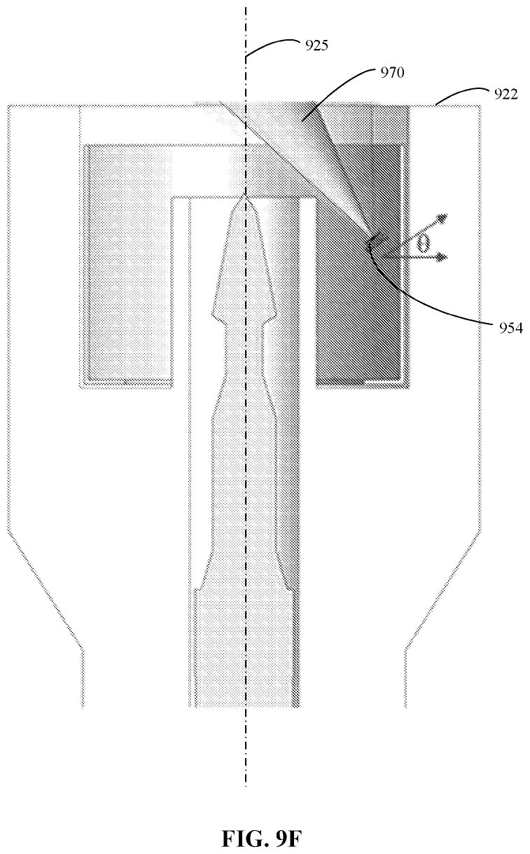

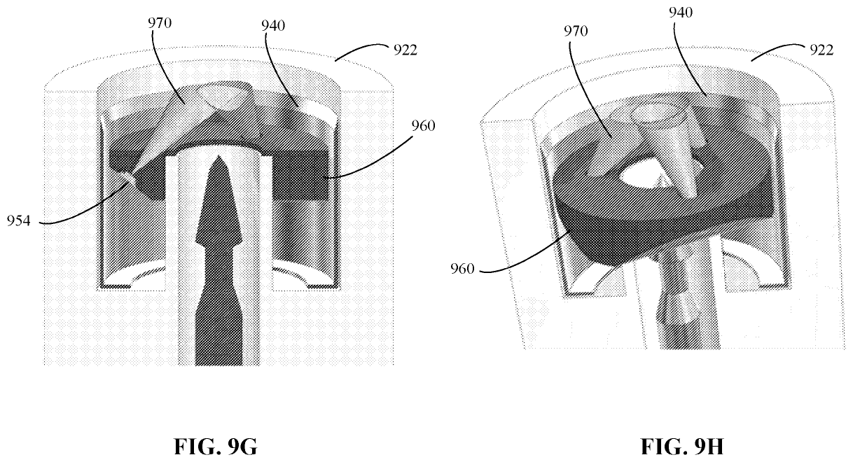

FIGS. 9A and 9B illustrate a stapler head 900 in accordance with some embodiments of the present invention. In some embodiments, the stapler head 900 or one or more components of the stapler head 900 may be formed with a distal end portion of a shaft assembly (e.g., the shaft assembly 110), permanently connected to the distal end portion of the shaft assembly, or removably attached to the distal end portion of the shaft assembly. For instance, in some embodiments, the stapler head 900 includes a crimp 910 configured for attaching the stapler head 900 to a distal end portion of a shaft assembly (e.g., the shaft assembly 110) and/or other functions. The stapler head 900 or one or more components of the stapler head 900 may be disposable or non-disposable.

In some embodiments, the stapler head 900 is configured to be engaged with an anvil (e.g., the anvil 130) as illustrated in FIGS. 9C-9E. Like the stapler heat 120 disclosed herein, the stapler head 900 can be operated to fire staples (e.g., the staples 211 illustrated in FIG. 2A) to suture together segments of a tubular organ (e.g., the segment 161 and the segment 162 of the tubular organ 160).

The stapler head 900 includes a stapling assembly 920, which may be configured substantially the same as or similar to the stapling assembly 220 disclosed herein. For instance, the stapling assembly 920 may be configured for operably supporting the circular staple cartridge 210, and may be formed with a distal end portion of a shaft assembly (e.g., the shaft assembly 110), permanently connected to the distal end portion of the shaft assembly, or removably attached to the distal end portion of the shaft assembly. In embodiments where the stapling assembly 920 is removably attached to the distal end portion of the shaft assembly, the stapling assembly 920 may be disposable or non-disposable.

In some embodiments, the stapling assembly 920 has a circumferential side wall 921 with a distal edge 922 that defines a staple line/plane (e.g., the staple line/plane 170 illustrated in FIGS. 1A and 1B). In some embodiments, the stapling assembly 920 further includes an interior base 923 as illustrated in FIG. 9E. The interior base 923 is located proximally away from the distal edge 922 (e.g., on the proximal side with respect to the distal edge 922). In some embodiments, the stapling assembly 920 further includes a tubular channel 926 coaxial with the circumferential side wall 921. Collectively, the circumferential side wall 921, the interior base 923, and the tubular channel 926 forms a recess 924 (e.g., a space) within the stapling assembly 920 between the distal edge 922 and the interior base 923.

In some embodiments, the stapler head 900 includes a trocar 930 configured for puncturing tissues of tubular organs and for engaging with an anvil (e.g., the anvil 130). The trocar 930 may be configured substantially the same as or similar to the trocar 230 disclosed herein. In some embodiments, the trocar 930 is connected to and supported by the stapling assembly 920, and/or other components. For instance, in some embodiments, at least a portion of the trocar is disposed in and supported by the crimp 910 and the tubular channel 926. In some embodiments, the trocar 930 is operably movable (e.g., can be operated to move, for instance, through the handle assembly 140) relative to the stapling assembly 920 along an axis 925 of the stapling assembly 920 between an extended position and a retracted position. In some embodiments, the crimp 910 and/or the tubular channel 926 are configured to guide the axial movement of the trocar 930. In some embodiments, the trocar 930 has a distal tip portion 931. When the trocar 930 is in the extended position, the distal tip portion 931 of the trocar 930 is exposed distally relative to the distal edge 922 of the stapling assembly 920, e.g., the distal tip portion 931 of the trocar 930 being positioned at the distal side of the distal edge 922 of the stapling assembly 920. When the trocar 930 is in the retracted position, the distal tip portion 931 of the trocar 930 is concealed proximally relative to the distal edge 922 of the stapling assembly 920, e.g., the distal tip portion 931 of the trocar 930 being positioned at the proximal side of the distal edge 922 of the stapling assembly 920.

In some embodiments, the stapler head 900 includes a tubular cutter 940 disposed in the recess 924 of the stapling assembly 920 and configured for cutting a tissue when operated. The tubular cutter 940 is operably movable (e.g., can be operated to move, for instance, through the handle assembly 140) relative to the stapling assembly 920 along an axis 925 of the stapling assembly 920. The tubular cutter 940 may be circular, and may be coaxial with the stapling assembly 920. The tubular cutter 940 may have a length (e.g., the axial dimension) in a range of from about 1 centimeter (cm) to about 2 cm, from about 1.5 cm to about 2.5 cm, or from about 2 cm to about 3 cm. The maximum distance that the tubular cutter 940 can be moved may be up to 3 cm, up to 3.5 cm, or up to 4 cm.

In some embodiments, the stapler head 900 includes a lighting piece 950 disposed in the recess 924 of the stapling assembly 920 and configured for providing illumination to a desired object or location (e.g., the trocar, the anvil, the working site). The lighting piece 950 includes one or more light sources 954, which may be the same as any light sources 240 disclosed herein. In some embodiments, the lighting piece 950 includes one or more segments 952 positioned at an oblique angle with respect to a particular reference, such as the axis 925 of the stapling assembly 920, the interior base 923, and/or the staple line/plane defined by the distal edge 922 of the stapling assembly 920. The one or more light sources 954 are disposed at the one or more segments 952. This allows for positioning the one or more light sources 954 at an oblique angle with respect to the particular reference such that the light 970 emitted from the one or more light sources 954 can be directed more effectively to a target object and/or a desired area (e.g., the anvil 130 and/or the working site) as illustrated in FIG. 9F. In some embodiments, the lighting piece 950 includes three oblique segments 952 each with a light source 954 as illustrated in FIG. 9B. However, it should be noted that this is by way of example and is non-limiting. The lighting piece 950 can be of other shapes, and can have any suitable number of oblique segments and any suitable number of light sources. The lighting piece 950 may be rigid or flexible. In some embodiments, the lighting piece 950 is made of silicone, polyimide, or other flexible material with a circuit embedded in the flexible material to power the one or more light sources 954. Alternatively, in some embodiments, the lighting piece 950 does not includes the one or more light sources 954, but is configured to power one or more light sources (e.g., LEDs) disposed at other locations (e.g., at the stapling assembly 920 or the frame 960).

In some embodiments, the stapler head 900 includes a frame 960 disposed in the recess 924 of the stapling assembly 920 and at a distal side of the lighting piece 950 (e.g., between the distal edge 922 of the stapling assembly 920 and the lighting piece 950 as illustrated in FIG. 9E). The frame 960 includes one or more guides 962 (e.g., holes, channels, slots, clear membranes) corresponding to the one or more light sources 954. For instance, in the embodiment illustrated in FIG. 9B, the frame 960 includes three guides 962, one for each light source 954. The one or more guides 962 are at an oblique angle with respect to the particular reference, such as the axis 925 of the stapling assembly 920, the interior base 923, and/or the staple line/plane defined by the distal edge 922 of the stapling assembly 920. The one or more guides 962 are configured for directing the light emitted from the one or more light sources 954 to a target object and/or a desired area (e.g., the anvil 130 and/or the working site) as illustrated in FIGS. 9G and 9H. In some embodiments, the frame 960 is configured for mounting the one or more light sources 954 and/or the lighting piece 940 so that each light source is adjacent to a corresponding guide 962.

The frame 960 may be made of stainless steel, ceramics, or a polymer like PEEK, UHMWPE, polycarbonate, or a PC-ABS blend. The frame 960 may also serve as a structure for mounting the one or more light sources 954 and/or the lighting piece 950. In some embodiments, the frame may be configured to allow adjustment of the vertical position and angle of the one or more light sources 954 to optimize and/or achieve a desired illumination (e.g., pattern and/or intensity of the light 970) at a desired location (e.g., on the staple plane). The adjustment may be conducted during the manufacturing of the device or before/during a surgical operation. In some embodiments, the vertical position and angle of the one or more light sources 954 may be adjusted, through the lighting piece 950 and/or the frame 960, to create a variety of patterns, such as a spotlight, crosshair, target, ring, or bullseye, on the staple plane. In some embodiments, the vertical position and angle of the one or more light sources 954 may be adjusted, through the lighting piece 950 and/or the frame 960, to optimize the visibility of the work site for a surgical camera positioned outside of the operation field. In some embodiments, the vertical position and angle of the one or more light sources 954 may be adjusted to optimize and/or maximize the light 970 leaving the stapler head 900, thereby enhancing illumination to the targeted object/site and/or visualization of the targeted object/site.

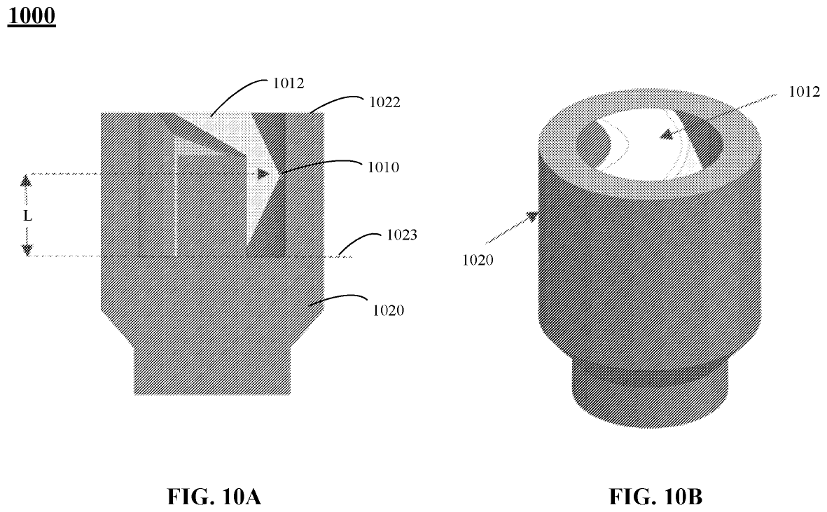

FIGS. 10A-10B illustrate an exemplary mechanism for optimizing and/or maximizing the visibility of the light leaving a stapler head 1000 in accordance with some embodiments of the present invention. The stapler head 1000 includes one or more light sources 1010 positioned within the stapler head 1000 at a vertical position “L” relative to an interior base 1023. The vertical position of the one or more light sources 1010 may be adjusted to optimize and/or maximize the light 1012 leaving the stapler head 1000, thereby enhancing illumination to the targeted object/site and/or visualization of the targeted object/site. The adjustment may be conducted during the manufacturing of the device or before/during a surgical operation.

The one or more light sources 1010 may be positioned to restrict the viewing angle of the light so as to focus the emitted light on a specific location (e.g., a specific spot within the staple plane 1022). The one or more light sources 1010 may be used in conjunction to create a specific light pattern and/or a visual image, such as a spotlight on the trocar, a crosshair pattern indicating the location of the trocar at the center, and/or a ring of light just inside the staple line. In some embodiments, the wavelength and/or intensity of a light source or light sources may be varied to help creation and/or control of the light pattern and/or visual image.

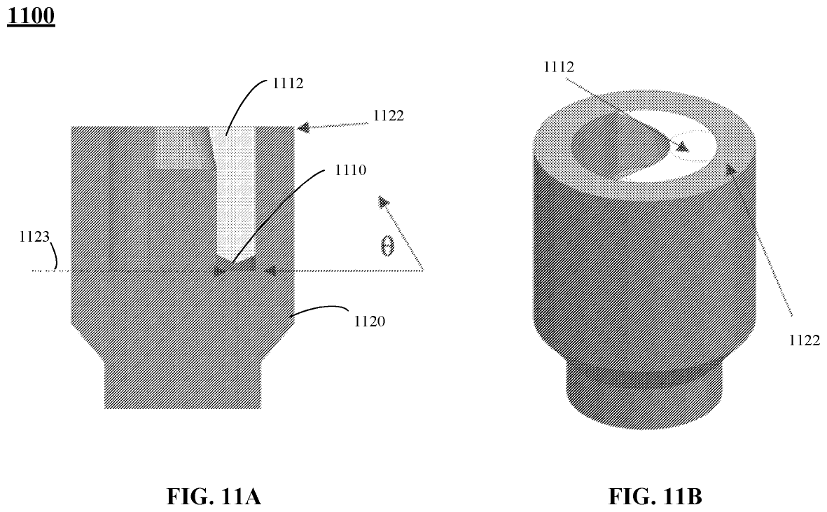

FIGS. 11A-10B illustrate an exemplary mechanism for optimizing and/or maximizing the visibility of the light leaving a stapler head 1100 in accordance with some embodiments of the present invention. The stapler head 1100 includes one or more light sources 1110 positioned on an interior base 1123 and at an angle with respect to the interior base 1123. The angle may be adjusted to optimize and/or maximize the light 1112 leaving the stapler head 1100, thereby enhancing illumination to the targeted object/site and/or visualization of the targeted object/site. The adjustment may be conducted during the manufacturing of the device or before/during a surgical operation.

The one or more light sources 1110 may be positioned to restrict the viewing angle of the light so as to focus the emitted light on a specific location (e.g., a specific spot within the staple plane 1122). The one or more light sources 1110 may be used in conjunction to create a specific light pattern and/or a visual image, such as a spotlight on the trocar, a crosshair pattern indicating the location of the trocar at the center, and/or a ring of light just inside the staple line. In some embodiments, the wavelength and/or intensity of a light source or light sources may be varied to help creation and/or control of the light pattern and/or visual image.

The surgical instruments and methods of the present disclosure are advantageous over existing devices. By employing light in one or more wavelength ranges (e.g., white light and/or NIR wavelengths) disposed at the trocar and/or the stapling assembly (e.g., around the circular staple line), the surgical instruments and methods of the present disclosure increases the visibility of the intended staple line/plane and trocar within tubular organs. This enables surgeons to correctly position the stapler head of the surgical instrument within tubular organs and accurately deploy the trocar of the stapler head transrectally. Accordingly, this allows for more consistent, reproducible, and standardized procedures to reduce the leak rates in colorectal anastomoses.

Illustration of Subject Technology as Clauses Various examples of aspects of the disclosure are described as numbered clauses (1, 2, 3, etc.) for convenience. These are provided as examples, and do not limit the subject technology.

Clause 1. A surgical instrument for treating a tubular organ of a patient, the surgical instrument comprising: a shaft assembly having a distal end portion and a proximal end portion; a stapling assembly disposed at the distal end portion of the shaft assembly and comprising a circumferential side wall having a distal edge to define a staple line; a trocar disposed at the distal end portion of the shaft assembly and operably movable relative to the stapling assembly along an axis of the stapling assembly between an extended position and a retracted position, wherein a distal tip portion of the trocar is exposed distally relative to the distal edge of the stapling assembly when the trocar is in the extended position and concealed proximally relative to the distal edge of the stapling assembly when the trocar in the retracted position; and one or more light sources disposed at the trocar, the stapling assembly or both for emitting light that is able to transmit through a wall of the tubular organ of the patient, thereby allowing a user of the surgical instrument to locate the trocar, the distal edge of the stapling assembly or both in the tubular organ of the patient by detecting light transmitted through the wall of the tubular organ.

Clause 2. The surgical instrument of Clause 1, wherein the tubular organ is a rectum or colon of the patient.

Clause 3. The surgical instrument of any preceding Clause, wherein the one or more light sources comprise at least one optic fiber, at least one LED, a polymer containing a near infrared fluorescent material, or any combination thereof.

Clause 4. The surgical instrument of any preceding Clause, wherein at least one light source in the one or more light sources emits light in a near-infrared light range.

Clause 5. The surgical instrument of any preceding Clause, wherein at least one light source in the one or more light sources is operable to emit light in any one of a plurality of wavelength ranges.

Clause 6. The surgical instrument of Clause 5, wherein the plurality of wavelength ranges comprises a first wavelength range and a second wavelength range different from the first wavelength range.

Clause 7. The surgical instrument of Clause 6, wherein the first wavelength range is in a white light range and the second wavelength range is in a near-infrared light range.

Clause 8. The surgical instrument of any preceding Clause, wherein at least one light source in the one or more light sources is operable to flash, change color, change intensity or any combination thereof.

Clause 9. The surgical instrument of any preceding Clause, wherein the light transmitted through the wall of the tubular organ of the patient is detected by a camera positioned outside of the tubular organ of the patient.

Clause 10. The surgical instrument of Clause 9, wherein the light detected by the camera is displayed to provide real-time visualization of where the trocar, the distal edge of the stapling assembly or both are located in the tubular organ of the patient.

Clause 11. The surgical instrument of any preceding Clause, wherein the one or more light sources comprise a plurality of light sources, wherein light sources in the plurality of light sources are spaced apart at known intervals to serve as markers for detection by a marker-based augmented reality system.

Clause 12. The surgical instrument of any preceding Clause, wherein: the stapling assembly further comprises an interior base located proximally away from the distal edge and a recess collectively formed by the interior base and the circumferential side wall; and the one or more light sources comprises: at least one first light source disposed at the distal tip portion of the trocar; at least one second light source disposed at a proximal base portion of the trocar; at least one third light source disposed on the interior base of the stapling assembly; at least one fourth light source disposed on an inner surface of the circumferential side wall of the stapling assembly; at least one fifth light source disposed on an outer surface of the circumferential all of the stapling assembly; or any combination thereof.

Clause 13. The surgical instrument of Clause 12, wherein the at least one first light source is different from the at least one second light source, the at least one third light source, the at least one fourth light source, or the at least one fifth light source.

Clause 14. The surgical instrument of any one of Clauses 12-13, wherein the at least one first light source comprises a first light source disposed at a very tip of the trocar.

Clause 15. The surgical instrument of any one of Clauses 12-14, wherein the trocar is made of a material transparent or translucent to the light emitted by the at least one first light source, the at least one second light source, or both.

Clause 16. The surgical instrument of Clause 15, wherein the material is a medical grade plastic.

Clause 17. The surgical instrument of Clause 16, wherein the medical grade plastic comprises acrylic (PMMA), transparent sulfone polymers, polymethyl methacrylate, PVC, polycarbonate, polyethylene terephthalate, polypropylene, polyethylene, styrene methyl methacrylate, or ionomer resin.

Clause 18. The surgical instrument of any one of Clauses 15-17, wherein the at least one first light source, the at least one second light source, or both are disposed inside of the trocar.

Clause 19. The surgical instrument of any one of Clauses 12-18, wherein the at least one third light source comprises a plurality of third light sources disposed circumferentially around the axis of the stapling assembly on the interior base of the stapling assembly.

Clause 20. The surgical instrument of Clause 19, wherein each of the plurality of third light sources is elongated radially with respect to the axis of the stapling assembly.

Clause 21. The surgical instrument of any one of Clauses 12-18, wherein the at least one third light source consists of a single third light source.

Clause 22. The surgical instrument of Clause 21, wherein the single third light source is a ring, encircling the axis of the stapling assembly.

Clause 23. The surgical instrument of any one of Clauses 12-22, wherein the at least one fourth light source comprises a plurality of fourth light sources disposed circumferentially around the axis of the stapling assembly on the inner surface of the circumferential side wall of the stapling assembly.

Clause 24. The surgical instrument of Clause 23, wherein each of the plurality of fourth light sources is elongated along the axis of the stapling assembly.

Clause 25. The surgical instrument of any one of Clauses 12-22, wherein the at least one fourth light source consists of a single fourth light.

Clause 26. The surgical instrument of Clause 25, wherein the single fourth light source is a ring, encircling the axis of the stapling assembly.

Clause 27. The surgical instrument of any one of Clauses 12-26, wherein the at least one fifth light source comprises a plurality of fifth light sources disposed circumferentially around the axis of the stapling assembly on the outer surface of the circumferential side wall of the stapling assembly.

Clause 28. The surgical instrument of Clause 27, wherein each of the plurality of fifth light sources is elongated along the axis of the stapling assembly.

Clause 29. The surgical instrument of any one of Clauses 12-26, wherein the at least one fifth light source consists of a single fifth light.

Clause 30. The surgical instrument of Clause 29, wherein the single fifth light source is a ring, encircling the axis of the stapling assembly.

Clause 31. The surgical instrument of any preceding Clause, further comprising: an anvil removably attached to the trocar.

Clause 32. The surgical instrument of Clause 31, wherein at least one light source in the one or more light sources changes color, changes intensity, flashes, or any combination thereof when the anvil is properly attached to the trocar.

Clause 33. The surgical instrument of Clause 32, further comprising: a sensor or a switch configured to detect the attachment of the anvil to the trocar.

Clause 34. The surgical instrument of any one of Clauses 31-33, wherein: the one or more light sources comprise a light source at the distal tip of the trocar, and the light source at the distal tip of the trocar is fully concealed by a complete attachment of the anvil to the trocar.

Clause 35. The surgical instrument of any preceding Clause, further comprising: a handle assembly disposed at the proximal end portion of the shaft assembly for operating the stapling assembly, the trocar, the one or more light sources, or any combination thereof.

Clause 36. The surgical instrument of Clause 35, wherein the operating of the stapling assembly, the trocar, the one or more light sources, or any combination thereof is powered or automatic.

Clause 37. The surgical instrument of Clause 36, wherein the handle assembly comprises a battery to power the one or more light sources, to aid the operation of the stapling assembly, to aid the operation of the trocar, or any combination thereof.

Clause 38. The surgical instrument of Clause 37, wherein the handle assembly is electrically connected to a power outlet to power the one or more light sources, to aid the operation of the stapling assembly, to aid the operation of the trocar, or any combination thereof.

Clause 39. The surgical instrument of Clause 35, wherein the operating of the stapling assembly, the trocar, the one or more light sources, or any combination thereof is manual via the handle assembly.

Clause 40. The surgical instrument of any one of Clauses 35-39, wherein the handle assembly comprises a rotatable knob connected to the trocar for extending or retracting the trocar.

Clause 41. The surgical instrument of any preceding Clause, wherein the stapling assembly, the trocar, and the one or more light sources collectively form a stapler head.

Clause 42. The surgical instrument of Clause 41, wherein the stapler head is removably attached to the distal end portion of the shaft assembly, disposable or both.

Clause 43. A method for using the surgical instrument of any preceding Clause to treat the tubular organ of the patient.

Clause 44. A method for treating a tubular organ of a patient, the method comprising: (A) inserting a stapler head of a surgical instrument into a first segment of the tubular organ, wherein the stapler head of the surgical instrument comprises a stapling assembly having a distal edge to define a staple line, a trocar operably movable relative to the stapling assembly along an axis of the stapling assembly, and one or more light sources disposed at the trocar and/or the stapling assembly to emit light capable of transmitting through a wall of the tubular organ; (B) detecting light transmitted through the wall of the tubular organ to locate the trocar, the distal edge of the stapling assembly or both in the first segment of the tubular organ; (C) determining, based on the detecting (B), whether the distal edge of the stapling assembly is positioned at a desired section of the first segment of the tubular organ; (D) moving, based on the determining (C), the stapler head of the surgical instrument until the distal edge of the stapling assembly is positioned at the desired section of the first segment of the tubular organ; (E) extending the trocar to puncture the desired section of the first segment of the tubular organ; (F) inserting an anvil into a second segment of the tubular organ, wherein the anvil comprises a connection portion protruding proximally out of a desired section of the second segment of the tubular organ; (G) attaching, aided at least in part by the light emitted from the one or more light sources, the connection portion of the anvil to the trocar; (H) drawing the anvil proximally toward the stapler head such that the desired sections of the first and second segments of the tubular organ are adjacent to each other; (I) stapling the desired sections of the first and second segments of the tubular organ together with staples of the stapling assembly; (J) cutting through the desired sections of the first and second segments of the tubular organ adjacent to the staples; and (K) removing the stapler head and anvil of the surgical instrument from the tubular organ.

Clause 45. The method of Clause 44, wherein the light detected in the detecting (B) is displayed to provide real-time visualization of where the trocar, the distal edge of the stapling assembly or both are located in the tubular organ of the patient.

Clause 46. The method of any one of Clauses 44-45, wherein the extending (E) is achieved by rotating a knob at a handle assembly of the surgical instrument in a first direction and the drawing (H) is achieved by rotating the knob in a second direction opposite to the first direction.

Clause 47. The method of any one of Clauses 44-46, wherein the inserting (F) is performed prior to the inserting (A).

Clause 48. The method of any one of Clauses 44-46, wherein the inserting (F) is performed concurrently with the inserting (A).

Clause 49. The method of any one of Clauses 44-46, wherein the inserting (F) is performed subsequent to the inserting (A).

Clause 50. The method of any one of Clauses 44-49, wherein at least one light source in the one or more light sources changes color, changes intensity, flashes or any combination thereof when the connection portion of the anvil is properly attached to the trocar.

Clause 51. The method of any one of Clauses 44-50, wherein the detecting (B) comprises: (i) visualization by the user of the surgical instrument, (ii) detection by a laparoscopic or robotic camera, or both.

Clause 52. The method of Clause 51, wherein the camera is operable in a white light mode, a near-infrared mode, or both.

### Terminologies and References Cited

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will be understood that the terms “left” or “right”, “top” or “bottom”, “lower” or “upper”, “interior” or “exterior”, “inward” or “outward” and etc. are used to describe features of the exemplary embodiments with reference to the positions of such features as displayed in the figures. It will be understood that, although the terms “first,” “second,” etc. may be used herein to describe various elements, these elements should not be limited by these terms. These terms are only used to distinguish one element from another. For example, a first element could be termed a second element, and, similarly, a second element could be termed a first element, without changing the meaning of the description, so long as the “first element” and the “second element” are renamed consistently.

As used herein, the term “and/or” as used herein refers to and encompasses any and all possible combinations of one or more of the associated listed items. It will be further understood that the terms “include”, “includes”, “including”, “comprise”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

The term “if” used herein is, optionally, construed to mean “when” or “upon” or “in response to determining” or “in response to detecting” or “in accordance with a determination that,” depending on the context. Similarly, the phrase “if it is determined” or “if [a stated condition or event] is detected” used herein is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

When a reference number is given an “i^{th}” denotation, the reference number refers to a generic component, set, or embodiment. For instance, a “unit i” refers to the i^{th} unit in a plurality of units.

All references cited herein are incorporated herein by reference in their entirety and for all purposes to the same extent as if each individual publication or patent or patent application was specifically and individually indicated to be incorporated by reference in its entirety for all purposes.

What is claimed is:

1. A surgical instrument for treating a tubular organ of a patient, the surgical instrument comprising:
a shaft assembly having a distal end portion and a proximal end portion;
a stapling assembly disposed at the distal end portion of the shaft assembly and comprising a circumferential side wall having a distal edge to define a staple line;
a trocar disposed at the distal end portion of the shaft assembly and operably movable relative to the stapling assembly along an axis of the stapling assembly between an extended position and a retracted position, wherein a distal tip portion of the trocar is exposed distally relative to the distal edge of the stapling assembly when the trocar is in the extended position and concealed proximally relative to the distal edge of the stapling assembly when the trocar in the retracted position, and
one or more light sources for emitting light that is able to transmit through a wall of the tubular organ of the patient, thereby allowing a user of the surgical instrument to locate the trocar, the distal edge of the stapling assembly or both in the tubular organ of the patient by detecting light transmitted through the wall of the tubular organ.

2. The surgical instrument of claim 1, wherein the one or more light sources comprise at least one optic fiber, at least one LED, a polymer containing a near infrared fluorescent material, or any combination thereof.

3. The surgical instrument of claim 1, wherein at least one light source in the one or more light sources emits light in a near-infrared light range.

4. The surgical instrument of claim 1, wherein at least one light source in the one or more light sources is operable to flash, change color, change intensity or any combination thereof.

5. The surgical instrument of claim 1, wherein the light transmitted through the wall of the tubular organ of the patient is detected by a camera positioned outside of the tubular organ of the patient.

6. The surgical instrument of claim 5, wherein the light detected by the camera is displayed to provide real-time visualization of where the trocar, the distal edge of the stapling assembly or both are located in the tubular organ of the patient.

7. The surgical instrument of claim 1, wherein the one or more light sources comprise a plurality of light sources, wherein light sources in the plurality of light sources are spaced apart at known intervals to serve as markers for detection by a marker-based augmented reality system.

8. The surgical instrument of claim 1, wherein:
the stapling assembly further comprises an interior base located proximally away from the distal edge and a recess collectively formed by the interior base and the circumferential side wall; and
the one or more light sources comprises:
at least one first light source disposed at the distal tip portion of the trocar;
at least one second light source disposed at a proximal base portion of the trocar;
at least one third light source disposed on the interior base of the stapling assembly;
at least one fourth light source disposed on an inner surface of the circumferential side wall of the stapling assembly;
at least one fifth light source disposed on an outer surface of the circumferential side wall of the stapling assembly;
at least sixth light source mounted on a frame in the recess of the stapling assembly; or
any combination thereof.

9. The surgical instrument of claim 8, wherein:
the at least one third light source, the at least one fourth light source, at least sixth light source, or any combination thereof is positioned at an angle with respect to the axis or the interior base of the stapling assembly to enhance illumination at a desired location.

10. The surgical instrument of claim 8, wherein:
the at least one fourth light source, at least sixth light source, or any combination thereof is positioned at a height with respect to the interior base of the stapling assembly to enhance illumination at a desired location.

11. The surgical instrument of claim 8, wherein:
the at least one fifth light source is mounted on a ring that is configured for removably fitting around the outer surface of the circumferential side wall of the stapling assembly.

12. The surgical instrument of claim 8, wherein the at least one first light source comprises a first light source disposed at a very tip of the trocar.

13. The surgical instrument of claim 8, wherein the trocar is made of a material transparent or translucent to the light emitted by the at least one first light source, the at least one second light source, or both, wherein the at least one first light source, the at least one second light source, or both are disposed inside of the trocar.

14. The surgical instrument of claim 8, wherein the at least one third light source (i) comprises a plurality of third light sources disposed circumferentially around the axis of the stapling assembly on the interior base of the stapling assembly, or (ii) consists of a single third light source encircling the axis of the stapling assembly.

15. The surgical instrument of claim 8, wherein the at least one fourth light source (i) comprises a plurality of fourth light sources disposed circumferentially around the axis of the stapling assembly on the inner surface of the circumferential side wall of the stapling assembly, or consists of a single fourth light encircling the axis of the stapling assembly.

16. The surgical instrument of claim 8, wherein the at least one fifth light source (i) comprises a plurality of fifth light sources disposed circumferentially around the axis of the stapling assembly on the outer surface of the circumferential side wall of the stapling assembly, or (ii) consists of a single fifth light encircling the axis of the stapling assembly.

17. The surgical instrument of claim 1, further comprising:
an anvil removably attached to the trocar.

18. The surgical instrument of claim 17, wherein at least one light source in the one or more light sources changes color, changes intensity, flashes, or any combination thereof when the anvil is properly attached to the trocar.

19. The surgical instrument of claim 18, further comprising:
a sensor or a switch configured to detect the attachment of the anvil to the trocar.

20. The surgical instrument of claim 1, further comprising:
a handle assembly disposed at the proximal end portion of the shaft assembly for operating the stapling assembly, the trocar, the one or more light sources, or any combination thereof.
